(19) United States
(12) Patent Application Publication
(10) Pub. No.: US 2014/0112929 A1
(43) Pub. Date: Apr. 24, 2014

(54) TUMOUR NECROSIS FACTOR RECEPTOR 1 ANTAGONISTS

(71) Applicant: Glaxo Group Limited, Brentford, Middlesex (GB)

(72) Inventors: Thil Dinuk Batuwangala, Cambridge (GB); Andrew Sanderson, Cambridge (GB); Armin Sepp, Cambridge (GB); Adriaan Allart Stoop, Cambridge (GB)

(73) Assignee: Glaxo Group Limited, Brentford, Middlesex (GB)

(21) Appl. No.: 14/126,568

(22) PCT Filed: Jun. 15, 2012
(86) PCT No.: PCT/EP2012/061489

§ 371 Date: Dec. 16, 2013

Related U.S. Application Data
(60) Provisional application No. 61/498,350, filed on Jun. 17, 2011.

(51) Int. Cl. C07K 16/28; A61K 39/395; G01N 33/566; C07K 16/18

(52) U.S. Cl. CPC C07K 16/2878; C07K 16/18; A61K 39/3955; G01N 33/566
USPC 424/139.1; 530/387.9; 436/501

(57) ABSTRACT

The invention relates to TNFR1 binding proteins, in particular those which are capable of preventing dimerisation of TNFR1 chains, and to their use in therapy.

The present invention relates to antagonists of tumour necrosis factor receptor 1 (TNFR1; p55), and to the use of such antagonists in therapy. The antagonists of the invention may be non-competitive antagonists, in that they are capable of antagonising TNFR1 via a mechanism which does not rely on the inhibition of the TNFα-TNFR1 interaction.

### BACKGROUND OF THE INVENTION

TNFR1 (p55) is a transmembrane receptor containing an extracellular region that binds ligand and an intracellular domain that lacks intrinsic signal transduction activity but can associate with signal transduction molecules. The crystal structure of soluble form of TNFR1 was first elucidated in complex with the TNFβ ligand (Banner et al., Cell, 73(3) 431-445 (1993)). The complex of TNFR1 with bound TNFβ showed three TNFR1 chains around a centrally-disposed trimeric TNFβ ligand. The three receptor chains are well separated from each other in this model and do not interact strongly. As TNFα is also active as a trimeric molecule, it was postulated that the TNFα-TNFR1 complex would be a closely similar structure. In this model, the three TNFR1 chains are clustered around the ligand in the receptor-ligand complex, and this clustering is considered to be a prerequisite to TNFR1-mediated signal transduction. In fact, multivalent agents that bind TNFR1, such as anti-TNFR1 antibodies, can induce TNFR1 clustering and signal transduction in the absence of TNF and are commonly used as TNFR1 agonists. (See, e.g., Belka et al., EMBO, 14(6):1156-1165 (1995); Mandik-Nayak et al., J. Immunol, 167:1920-1928 (2001).) Accordingly, multivalent agents that bind TNFR1 are generally not effective antagonists of TNFR1 even if they block the binding of TNFα to TNFR1.

The extracellular region of human TNFR1 comprises a thirteen amino acid amino-terminal segment (amino acids 1-13 of SEQ ID NO:1), four cysteine rich domains, Domain 1 (amino acids 14-53 of SEQ ID NO:1), Domain 2 (amino acids 54-97 of SEQ ID NO:1), Domain 3 (amino acids 98-138 of SEQ ID NO:1), and Domain 4 (amino acids 139-167 of SEQ ID NO:1)), which are followed by a membrane-proximal region (amino acids 168-182 of SEQ ID NO:1). Domains 2 and 3 make contact with bound ligand (TNFβ, TNFα). (See, Banner (Id.) and Loetscher et al., Cell 61(2) 351-359 (1990)).

TNFR1 is also capable of dimerisation in the absence of ligand (Naismith et al. JBC 22:13303-13307 (1995), and Naismith et al., Structure 4:1251-1262 (1996)). The authors describe various dimeric forms of the receptor, and identify the key residues involved in those interactions. Chan (Chan et al. Science, 288:235-2354 (2000)) and Deng (Deng et al., Nature Medicine, doi: 10.1038/nm1304 (2005)) later identified a region within domain 1 of TNFR1, referred to as the pre-ligand binding assembly domain or PLAD (amino acids 1-53 of SEQ ID NO:1), as responsible for receptor chain association. Chan et al. suggest that PLAD is distinct from the ligand binding domain, but is responsible for the self-association of TNFR1 prior to ligand binding, and is “necessary and sufficient” for the assembly of trimeric TNFR1 complexes that bind TNFα.

TNFR1 is shed from the surface of cells in vivo through a process that includes proteolysis of TNFR1 in Domain 4 or in the membrane-proximal region (amino acids 168-182 of SEQ ID NO:1; amino acids 168-183 of SEQ ID NO:2), to produce a soluble form of TNFR1. Soluble TNFR1 retains the capacity to bind TNFα, and thereby functions as an endogenous inhibitor of the activity of TNFα.

The consequences of TNFR2 activation are less well characterised than those of TNFR1, but are considered to be primarily responsible for mediating cell proliferation, migration and survival, as well as promoting tissue repair and angiogenesis (Kim et al., J. Immunol. 173 4500-4509 (2004), Bradley, J. Pathol. 214(2) 149-160). Blockade of TNF-mediated host defence can increase the risk of bacterial or viral infection, or of development of lymphoma (Mukai et al. Sci. Signal. 3, Ra83 (2010)). The specific blocking of TNFR1 signalling is considered to be a promising approach which will minimize the side effects of TNFα blockade.

Although soluble versions of PLAD have been shown to block binding of TNFα to TNFR1, without binding to TNFα, this effect was not necessarily specific to TNFR1 (Deng et al. (Id.)). Deng et al. also proposed a model of TNFR1 receptor trimerisation in which PLAD is involved in the formation of a trimeric receptor complex prior to ligand binding. The authors also acknowledge that the PLAD proteins had an extremely short half-life, and that it would be advantageous to provide agents which can mimic the effect of PLAD but require less frequent dosing.

WO2006038027, WO2008149144, WO2008149148, WO2010094720, WO2011006914 and WO2011051217 describe anti-TNFR1 immunoglobulin single variable domains. These documents also describe the use of such immunoglobulin single variable domains for the treatment and/or prevention of conditions mediated by TNFα. Certain immunoglobulin single variable domains described in these applications bind to an epitope on TNFR1 which is distinct from the epitope that is engaged by the natural TNFα ligand, and prevent signalling through TNFR1. Molecules with such characteristics are herein termed non-competitive inhibitors of TNFR1.

It would be desirable to provide additional TNFR1 antagonists and products comprising these. The aim of these would be to provide improved therapeutics for the treatment and/or prophylaxis of TNFR1-mediated conditions and diseases in humans or other mammals. The various aspects of the present invention meet these desirable characteristics.

### SUMMARY OF THE INVENTION

In a first aspect, the invention provides a TNFR1 binding protein, wherein the TNFR1 binding protein binds to an epitope on TNFR1 (SEQ ID NO:1), wherein the epitope comprises or consists of one or more residues selected from: Q17, G18, K19, T31, K32, C33, H34, K35, G36, T37, G47, Q48, D49, E54, E64, V90, V91, H126, L127, Q130, Q133, V136, T138 and L145 of SEQ ID NO:1.

In another aspect, the invention provides a TNFR1 binding protein, wherein the TNFR1 binding protein binds to an epitope on TNFR1 (SEQ ID NO:1), wherein the epitope comprises or consists of one or more residues selected from: Q17, G18, K19, T31, K32, C33, H34, K35, G36, T37, G47, Q48, D49, E54, E64, V90, V91, L127, Q130, Q133 and V136 of SEQ ID NO:1.

In another aspect, the invention provides a TNFR1 binding protein, wherein the TNFR1 binding protein binds to an epitope on TNFR1 (SEQ ID NO:1), wherein the epitope comprises or consists of one or more residues selected from: Q17, G18, K19, T31, K32, C33, H34, K35, G36, T37, G47, Q48, D49, E54, E64, V90, V91, H126, L127, Q130, Q133, V136, T138 and L145 of SEQ ID NO:1, on the proviso that, if the TNFR1 binding protein binds to an epitope that comprises or consists of one or more of residues H126, T138 and L145, the TNFR1 binding protein is not an immunoglobulin single variable domain.

In an embodiment, the TNFR1 binding protein is an antibody, single variable domain, a domain antibody, an antigen binding or immunologically effective fragment of an antibody, including a Fab, F(ab′)₂, Fv, disulphide linked Fv, scFv, closed conformation multispecific antibody, disulphide-linked scFv, diabody or Tandab™, or a protein construct capable of binding specifically to TNFR1. In a particular embodiment, the TNFR1 binding protein is an immunoglobulin single variable domain.

The TNFR1 binding protein may bind monovalently to TNFR1.

In an embodiment, the TNFR1 binding protein is an antagonist of TNFR1. The TNFR1 binding protein may be a non-competitive antagonist of TNFR1, in that the binding of TNFR1 binding protein does not antagonise the binding of TNFα ligand to the TNFR1.

In an embodiment, the TNFR1 binding protein binds to an epitope on TNFR1, wherein the epitope comprises or consists of at least one of residues: Q17, G18, K19, T31, K32, C33, H34, K35, G36, T37, G47, Q48, D49, E54, E64, V90, V91, L127, Q130, 0133 and V136 of SEQ ID NO:1.

In an embodiment, the TNFR1 binding protein binds to an epitope on TNFR1, wherein the epitope comprises or consists of one or more residues selected from: Q17, G18, K19, T31, K32, C33, H34, K35, G36, T37, G47, Q48 and D49 of SEQ ID NO:1.

In an embodiment, the TNFR1 binding protein binds to an epitope on TNFR1, wherein the epitope comprises or consists of one or more residues selected from: E54, E64, V90 and V91 of SEQ ID NO:1.

In an embodiment, the TNFR1 binding protein binds to an epitope on TNFR1, wherein the epitope comprises or consists of one or more residues selected from: H126, L127, Q130, Q133, V136 and T138 of SEQ ID NO:1. In an embodiment, the TNFR1 binding protein binds to an epitope on TNFR1, wherein the epitope comprises or consists of four or more residues selected from: H126, L127, Q130, Q133, V136 and T138 of SEQ ID NO:1. In an embodiment, the TNFR1 binding protein binds to an epitope on TNFR1, wherein the epitope comprises or consists of one or more residues selected from: H126, L127, Q130, Q133, V136, T138 and L145 of SEQ ID NO:1. In an embodiment, the TNFR1 binding protein binds to an epitope on TNFR1, wherein the epitope comprises or consists of one or more residues selected from: L127, Q130, Q133 and V136 of SEQ ID NO:1.

In an embodiment, the TNFR1 binding protein binds to an epitope on TNFR1, wherein the epitope comprises or consists of residue L145 of SEQ ID NO:1. In an embodiment, the TNFR1 binding protein binds to an epitope on TNFR1, wherein the epitope comprises or consists of residue L145 and at least one of residues L127, Q130 and V136 of SEQ ID NO:1.

In any aspect of the invention or embodiment herein described, in one embodiment the TNFR1 binding protein binds to an epitope on TNFR1, wherein the epitope does not comprise at least one of residues selected from: T124, C139, H140, A141, F143, F144, E161, L165, L167, P168 and Q169 of SEQ ID NO:1.

In another aspect, the invention provides an anti-TNFR1 binding protein which binds to an epitope within TNFR1 and prevents dimerisation of TNFR1, wherein the epitope does not comprise or require residues H126, T138 or L145.

In one embodiment, the TNFR1 binding protein is not an immunoglobulin single variable domain. In another aspect, the invention provides a TNFR1 binding protein, which competes for binding to TNFR1 (SEQ ID NO:1) with Dom1h-574-208 (SEQ ID NO:2), on the proviso that the TNFR1 binding protein is not an immunoglobulin single variable domain.

In another aspect, the invention provides a TNFR1 binding protein as described herein, wherein the TNFR1 binding protein comprises a second binding specificity for an antigen other than TNFR1. In an embodiment, the antigen other than TNFR1 is human serum albumin.

In another aspect, the invention provides a multispecific ligand, comprising a TNFR1 binding protein as described herein and a binding protein that specifically binds to an antigen other than TNFR1. In an embodiment, the antigen other than TNFR1 is human serum albumin.

In another aspect, the invention provides a TNFR1 binding protein which is an antagonist of TNFR1 dimerisation, wherein the TNFR1 binding protein binds to an epitope comprising or consisting of one or more of residues selected from: Q17, G18, K19, T31, K32, C33, H34, K35, G36, T37, G47, Q48, D49, E54, E64, V90, V91, H126, L127, Q130, Q133, V136, T138 and L145 of SEQ ID NO:1.

In an embodiment, the TNFR1 binding protein is a non-competitive TNFR1 antagonist. In an embodiment, the TNFR1 binding protein binds to an epitope comprising or consisting of one or more of residues: E54, E64, V90 and V91, H126, L127, Q130, Q133, V136, T138 and L145 of SEQ ID NO:1. In an embodiment, the TNFR1 binding protein binds to an epitope comprising or consisting of one or more of residues E54, E64, V90 and V91, L127, Q130, Q133 and V136 of SEQ ID NO:1.

In a related aspect, the invention provides a method for the treatment or prophylaxis of an inflammatory condition in a patient comprising administering an antagonist of TNFR1 dimerisation to the patient. In these and other aspects of the invention, optionally the TNFR1 binding protein is not a domain antibody.

In another aspect, the invention provides a TNFR1 antagonist comprising a TNFR1 binding protein or a multispecific ligand according to the invention.

In another aspect, the invention provides a composition comprising a TNFR1 binding protein according to the invention in a physiologically acceptable carrier.

The invention also provides a method for the treatment or prophylaxis of an inflammatory condition in a patient, the method comprising administering the TNFR1 binding protein according to the invention to the patient.

In another aspect, the invention provides a method of preventing amplification of TNFR1 signal transduction, comprising the steps of providing a TNFR1 binding protein according to the invention under conditions suitable to allow it to bind to TNFR1, thereby preventing the multimerisation of TNFα-TNFR1 trimeric complexes.

In another aspect, the invention provides a method of preventing dimerisation of TNFR1, comprising the steps of providing a TNFR1 binding protein according to the invention under conditions suitable to allow it to bind to TNFR1, thereby preventing the TNFR1 chain from dimerisation. The conditions may be physiologically acceptable conditions.

In an embodiment, the anti-TNFR1 binding protein is a non-competitive antagonist of TNFR1.

The invention also provides a method for the treatment or prophylaxis of an inflammatory condition in a patient, the method comprising administering to the patient an inhibitor of the amplification of TNFR1 signal transduction.

The invention also provides a method for the treatment or prophylaxis of an inflammatory condition in a patient, the method comprising administering to the patient an inhibitor of TNFR1 dimerisation.

In another aspect, there is provided a method of screening for non-competitive antagonists of TNFR1, comprising the steps of providing a plurality of TNFR1 binding proteins, determining the ability of said TNFR1 binding proteins to antagonise TNFR1 signalling, determining the ability of said TNFR1 binding proteins to disrupt the binding of TNFR1 to TNFα, and selecting those TNFR1 binding protein which antagonise TNFR1 but which do not disrupt the binding of TNFR1 to TNFα.

Receptor binding assays and inhibitory assays (to assess the functional response to TNFα) are well known to the skilled person. Reference may also be made to the methods described in Example 1.

In another aspect, there is provided a method of screening for non-competitive antagonists of TNFR1, comprising the steps of determining the epitope of a TNFR1 antagonist, and selecting antagonists which have an epitope comprising one or more amino acid residues of TNFR1 (SEQ ID NO:1) selected from: Q17, G18, K19, T31, K32, C33, H34, K35, G36, T37, G47, Q48, D49, E54, E64, V90, V91, H126, L127, Q130, 0133, V136, T138 and L145. The antagonist may be an TNFR1 binding protein.

In an embodiment, the antagonists are selected from those which have an epitope comprising one or more of residues: E54, E64, V90 and V91, H126, L127, Q130, 0133, V136, T138 and L145 of SEQ ID NO:1, more particularly residues E54, E64, V90 and V91, L127, Q130, 0133 and V136 of SEQ ID NO:1.

Also provided is a non-competitive antagonist of TNFR1 obtained by such screening processes.

The mechanism of action of TNFR1 antagonists (i.e. those which operate via non-competitive inhibitors of TNFR1 dimerisation) which is identified herein is believed to be applicable to other members of the TNF receptor superfamily. These receptors are structurally similar to TNFR1, and therefore prevention of dimerisation exemplified by DOM1h-574-208 would be predicted to antagonise those family members in a similar manner. Therefore, all aspects herein described are considered to be correspondingly applicable to other members of the TNFR superfamily.

Accordingly, binding proteins which have epitopes which comprise or consist of corresponding residues to those identified herein (i.e. those involved in dimerisation of the TNFR superfamily member, in particular those residues in the membrane-proximal cysteine-rich domain 4 (and thus involved in multimerisation of the receptor ligand complexes) are also provided by the present invention. TNFR superfamily members are described by Locksley et al. Cell (2001) 104:487-501, and include NGFR, Troy, EDAR, XEDAR, CD40, DcR3, FAS, OX40, AITR, CD30, HveA, 4-IBB, TNFR2, DR3, CD27, LTβr, RANK, TACI, BCMA, DR6, DR4, DR5, DcR1 and DcR2.

### BRIEF DESCRIPTION OF THE FIGURES

FIG. 1 (a) is a graph showing the results of a TNFα receptor binding assay (RBA), comparing the effect of a non-competitive TNFR1 binding protein (DOM1h-574-208) and a competitive TNFR1 binding protein (DOM1h-131-206) on the ability of TNFα to bind TNFR1. FIG. 1 (b) is a graph showing the results of a TNFα functional assay, showing that both competitive and non-competitive TNFR1 binding proteins are capable of inhibiting TNFα signal transduction.

FIG. 2 (a) is a photograph of DOM1h-574-208-TNFR1-TNFα crystals; FIG. 2 (b) is an SDS-PAGE analysis of complex.

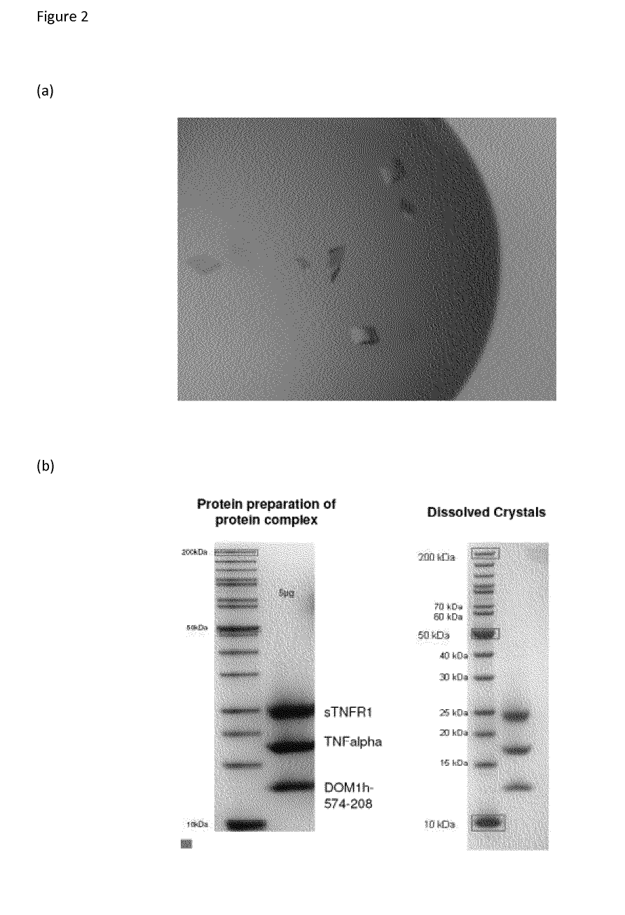

FIG. 3 shows the elucidated TNFR1-TNFα crystal structure, with DOM1h-574-208 bound thereto. This complex could form on the cell surface, with three DOM1h-574-208 molecules on the outside of the trimeric complex, and the TNFα trimer centrally disposed (FIG. 3).

FIG. 4 shows the binding sites of TNFα and DOM1h-574-208 on a single TNFR1 chain. TNFR1 is orientated in such a way that domain 1 is at the apex. The uppermost right hand panel highlights the TNFα binding site in black. The lowermost right hand panel highlights the epitope of DOM1h-574-208.

FIG. 5 upper panel is a graphical representation comparing the DOM1h-574-208 epitope with the TNFR1 dimerisation interface (both shown in black). The lower four panels show the DOM1h-574-208-TNFR1 epitope interactions which overlap with TNFR1 dimerisation interface.

FIG. 6 (a)-(e) is a graphical representation of the step-wise multimerisation of TNFα-TNFR1.

FIG. 7 (a) is a graphical representation of how the TNFR1 dimerisation inhibitors of the present invention prevent multimerisation of TNFα-TNFR1 trimers. FIG. 7(b) is a schematic representation of TNFR1 interacting with ligands in the absence of TNFα (panel A) and in the presence of TNFα(panel B).

### DETAILED DESCRIPTION OF THE INVENTION

The prevailing TNF-α signalling paradigm is built on the ‘trimerisation hypothesis’ whereby interaction between the intracellular domains of three ligand-cross-linked receptor molecules is necessary and sufficient to initiate signalling (Banner, Cell 1993 7; 73(3):431-45). The identification of a parallel TNFR1 dimer structure evolved this hypothesis to the ‘extended network hypothesis’ in which clusters of receptor homodimers and TNF-α homotrimers create an expandable arrangement of TNFR1/TNF-α complexes, possibly amplifying the signal (Naismith, 1995, supra).

In support of this network hypothesis, a role for ligand-independent receptor assembly was provided by the identification and requirement of the pre-ligand assembly domain (PLAD), which constitutes CRD1, for signalling (Chan, 2000, supra). The prevalence of TNFR1 to exist as homodimers on the cell surface was demonstrated in an elegant chemical cross-linking and immunoprecipitation study by Boschert (Boschert Cell Signal. 2010 22(7):1088-96), who also concluded that TNFR1 does not require engagement with all three TNF-α molecules in the trimer to signal. A similar conclusion could be reached from the observation that bivalent TNFR1 cross-linking at the ligand binding site by an agonistic mAb is sufficient to trigger signalling while a monomeric Fab fragment, derived from the same mAb, is inactive (Engelmann J Biol Chem. 1990 25; 265(24):14497-504).

The results described herein though indicate that disrupting the TNFR1 dimers by binding of a monovalent TNFR1 binding protein (a domain antibody) in the TNFR1 homodimer interface is sufficient to inhibit signalling, even though TNF-α is still able to recruit three receptors as demonstrated in the crystal structure. This leads us to propose a minimal TNF-α/TNFR1 signalling unit consisting of a TNF-α trimer cross-linking at least two pre-formed TNFR1 homodimers, present in a parallel structure as described by Naismith (Naismith, 1995 supra), thereby bringing together four intracellular TNFR1 death domains in a configuration that can signal. Given that receptor dimers in the absence of TNF-α are inactive, we suggest that any interactions between death domains in homodimerised TNFR1 are not involved in signalling. Similarly insufficient are interactions between the death domains of neighbouring monomeric TNFR1 nucleated around TNF-α, as in the presence of the domain antibody. Hence, we propose signalling to occur from the death domain of a non-ligand-contact TNFR1 to that of a ligand-contact TNFR1 subunit of the second TNFR1 homodimer and vice versa. Conceptually, a highly comparable receptor arrangement would be achieved through bivalent engagement with a mAb, explaining the prevalence of mAb-induced agonism of TNFR1 receptors.

This model would also help explain the surprising results (not shown) obtained with a bivalent format of the same domain antibody conjugated to an Fc region. This molecule would cross-link TNFR1 monomers in an organisation reminiscent of the homodimer, an organisation itself insufficient to induce signalling, therefore not resulting in agonism. Similarly, the bivalent domain antibody-Fc molecule does not inhibit formation of the minimal signalling unit in the presence of TNF-α, maintaining receptors in a dimeric organisation, and consequently lacks antagonistic activity. The lack of functional impact of bivalent engagement with CRD4 would help rationalise why the unique mechanism of mAbs binding this epitope might not have been recognised previously.

These observations suggest a novel approach to TNF pathway antagonism, enabling segregation at the receptor level. The domain antibody described herein, and TNFR1 binding proteins which bind to CRD4 of TNFR1 and prevent receptor dimerisation in the same manner offer promising alternative therapeutic approaches to the anti-TNF approach. Given the dominant contribution of TNFR1 to most inflammatory processes (Bradley J Pathol. 2008 214(2):14) and the suggested beneficial contributions of TNFR2 to immuno-suppression (Chen Immunology 2011 133(4):426-33), specific inhibition of TNFR1, instead of TNF-α, might provide treatment benefits to patients in comparison to the anti-TNFα approach. In a similar manner, proteins which bind to CRD4 of other members of the TNFR superfamily would be predicted to offer novel therapeutic approaches for antagonising the receptor.

Within this specification the invention has been described, with reference to embodiments, in a way which enables a clear and concise specification to be written. It is intended and should be appreciated that embodiments may be variously combined or separated without parting from the invention.

Unless defined otherwise, all technical and scientific terms used herein have the same meaning as commonly understood by one of ordinary skill in the art (e.g., in cell culture, molecular genetics, nucleic acid chemistry, hybridization techniques and biochemistry). Standard techniques are used for molecular, genetic and biochemical methods (see generally, Sambrook et al., Molecular Cloning: A Laboratory Manual, 2d ed. (1989) Cold Spring Harbor Laboratory Press, Cold Spring Harbor, N.Y. and Ausubel et al., Short Protocols in Molecular Biology (1999) 4^{th} Ed, John Wiley & Sons, Inc. which are incorporated herein by reference) and chemical methods.

As used herein, the term “TNFR1 binding protein” refers to antibodies and other protein constructs, such as domains or DARPins (designed ankyrin repeat proteins), which are capable of binding to TNFR1. TNFR1 binding proteins may be antagonists of TNFR1, or may be agonists of TNFR1. Antagonists of TNFR1 may be non-competitive antagonists of TNFR1.

The term “antibody” is used herein in the broadest sense to refer to molecules with an immunoglobulin-like domain and includes monoclonal, recombinant, polyclonal, chimeric, human, humanised, multispecific including bispecific antibodies, and heteroconjugate antibodies; a single variable domain (e.g. V_{H}, V_{HH}, V_{L}, domain antibody (dAb™)), antigen binding fragments including Fab, F(ab′)₂, Fv, disulphide linked Fv, scFv, disulphide-linked scFv, diabody TANDABS™, etc. and modified versions of any of the foregoing (for a summary of alternative “antibody” formats see Holliger and Hudson, Nature Biotechnology, 2005, Vol 23, No. 9, 1126-1136).

The phrase “single variable domain” refers to a folded polypeptide domain comprising sequences characteristic of antibody variable domains. It therefore includes complete antibody variable domains such as V_{H}, V_{HH}, V_{L} and modified antibody variable domains, for example, in which one or more loops have been replaced by sequences which are not characteristic of antibody variable domains, or antibody variable domains which have been truncated or comprise N- or C-terminal extensions, as well as fragments of variable domains which retain at least the binding activity and specificity of the full-length domain. A single variable domain is capable of binding an antigen or epitope independently of other variable regions or domains. A single variable domain may be a human single variable domain, but also includes single variable domains from other species such as rodent (for example, as disclosed in WO 00/29004), nurse shark and Camelid V_{HH} dAbs™. Camelid V_{HH} are immunoglobulin single variable domains that are derived from species including camel, llama, alpaca, dromedary, and guanaco, which produce heavy chain antibodies naturally devoid of light chains. Such VHH domains may be humanised according to standard techniques available in the art, and such domains are considered to be “single variable domains”. As used herein V_{H} includes camelid V_{HH} domains.

An single variable domain can be present in a format (e.g., homo- or hetero-multimer) with other variable regions or variable domains where the other regions or domains are not required for antigen binding by the single variable domain (i.e., where the immunoglobulin single variable domain binds antigen independently of the additional variable domains). In one embodiment, in any aspect described herein, the TNFR1 binding protein is not an immunoglobulin single variable domain.

A “domain” is a folded protein structure which has tertiary structure independent of the rest of the protein. Generally, domains are responsible for discrete functional properties of proteins, and in many cases may be added, removed or transferred to other proteins without loss of function of the remainder of the protein and/or of the domain.

As used herein, “functional” describes a polypeptide or peptide that has biological activity, such as specific binding activity. For example, the term “functional polypeptide” includes an antibody or antigen-binding fragment thereof that binds a target antigen through its antigen-binding site.

As used herein, “antibody format”, “formatted” or similar refers to any suitable polypeptide structure in which one or more antibody variable domains can be incorporated so as to confer binding specificity for antigen on the structure. A variety of suitable antibody formats are known in the art, such as, chimeric antibodies, humanized antibodies, human antibodies, single chain antibodies, bispecific antibodies, antibody heavy chains, antibody light chains, homodimers and heterodimers of antibody heavy chains and/or light chains, antigen-binding fragments of any of the foregoing (e.g., a Fv fragment (e.g., single chain Fv (scFv), a disulfide bonded Fv), a Fab fragment, a Fab′ fragment, a F(ab′)₂ fragment), a single variable domain (e.g., a dAb, V_{H}, V_{HH}, V_{L}), and modified versions of any of the foregoing (e.g., modified by the covalent attachment of polyethylene glycol or other suitable polymer or a humanized V_{HH}).

An antigen binding fragment may be provided by means of arrangement of one or more CDRs on non-antibody protein scaffolds such as a domain. The domain may be a domain antibody or may be a domain which is a derivative of a scaffold selected from the group consisting of DARPin, CTLA-4, lipocalin, SpA, an Affibody, an avimer, GroEl, transferrin, GroES and fibronectin/adnectin, which has been subjected to protein engineering in order to obtain binding to an antigen, such as TNFR1, other than the natural ligand.

An antigen binding fragment or an immunologically effective fragment may comprise partial heavy or light chain variable sequences. Fragments are at least 5, 6, 8 or 10 amino acids in length. Alternatively the fragments are at least 15, at least 20, at least 50, at least 75, or at least 100 amino acids in length.

The term “epitope” as used herein has its regular meaning in the art. Essentially, an epitope is a protein determinant capable of specific binding to an antigen binding protein, such as a TNFR1 binding protein. Epitopes usually consist of chemically active surface groupings of molecules such as amino acids or sugar side chains and usually have specific three dimensional structural characteristics, as well as specific charge characteristics.

The term “binding” or “specific binding” used herein in the context of “binding to an epitope comprising residue X” is given its normal meaning in the art. Identifying the amino acid residues which make up an epitope on a target antigen—i.e. those residues involved in the “binding” interaction between binding protein and target antigen is routine in the art. An epitope may be determined by, for example, competition assays with monoclonal antibodies (or other antigen binding proteins) of which the binding epitope is known, on e.g. Biacore, peptide mapping, site-directed mutagenesis (e.g. alanine scanning mutagenesis), hydrogen-deuterium exchange mass-spectrometry, x-ray crystallography. For example, an epitope may be defined accurately by mapping those residues in the antigen which are determined by X-ray crystallography to be within 4.0 Å (i.e. 4.0 Å or less than 4.0 Å) of a residue in the antigen binding protein.

As used herein, the term “antagonist of Tumor Necrosis Factor Receptor 1 (TNFR1)”, “TNFR1 antagonist” or the like refers to an agent (e.g., a molecule, a compound) which binds TNFR1 and can inhibit a (i.e., one or more) function of TNFR1. For example, an antagonist of TNFR1 can inhibit signal transduction mediated through TNFR1. Antagonists of TNFR1 include those which partially, but not completely, inhibit a function of TNFR1 (herein referred to as “partial antagonists” of TNFR1). For instance, the antagonists described herein may partially, but not completely, abrogate signal transduction mediated through TNFR1 (e.g. may abrogate signal transduction substantially completely at a first concentration of TNFa, but only partially at a second, higher concentration).

Antagonists which partially inhibit TNFR1 are described in WO20110066914, the content of which is hereby incorporated in its entirety. Non-competitive TNFR1 binding proteins have been observed to display a decreased level of inhibition at increasing TNFα concentrations (WO2011006914), suggesting that they would be partial inhibitors of TNFα when high concentrations of TNFα are present. Consequently at high TNFα concentrations this class of inhibitors would leave residual TNFα signalling uninhibited. They offer potential advantages vis-a-vis complete inhibition of the effects of TNFα, as they do not completely inhibit all TNFα, but only the excess amount of TNFα found during chronic inflammation, e.g. in arthritis.

Excess TNFα production is one of the causes of the pathogenesis of inflammatory disease such as rheumatoid arthritis and inhibition of TNFα using anti-TNFα antibodies has been highly effective in the treatment of patients. However, TNFα also plays an important role in host immune defence by increasing phagocytosis by macrophages and enhancing mycobacterial killing in concert with IFNγ. The importance of this additional activity of TNFα is highlighted by the epidemiological evidence that individuals treated with TNFα inhibitors have an increased risk for the development of infections in the respiratory tract, in particular the reactivation of tuberculosis. Because of this dual role for TNFα, the incomplete inhibition of TNFα might be beneficial for reducing the susceptibility to infections. Most extensive modelling of the effects of residual free soluble TNFα on bacterial load was published by Marino et al (Marino et al., PLoS Comput Biol. 2007 October; 3(10):1909-24). The models disclosed in this publication suggest that only a very small amount of soluble TNFα is required for control of the infection. In the discussion Marino et al reiterate their major finding: ‘ . . . that anti-TNF therapy will likely lead to numerous incidents of primary TB if used in areas where exposure is likely, and that sTNF—even at very low levels—is essential for control of infection.’ Very similar conclusions were reached by Guler et al (Guler et al, Infect Immun. 2005 Jun. 1; 73(6):3668-76), in a study comparing the effects of total and partial neutralisation of TNFα on cell-mediated immunity to Mycobacterium bovis BCG infection in mice. In this experimental study, regulation of TNFα levels was accomplished using transgenic mice expressing TNFR1 at varying levels. They conclude: ‘ . . . total neutralisation of TNF led to increased susceptibility [to BCG infection], whereas partial TNF inhibition resulted in enhanced granuloma formation and macrophage activities.’ These results were mimicked by Plessner et al (Plessner et al. J Infect Dis. 2007 Jun. 1; 195(11):1643-50) in a chronic murine tuberculosis model comparing a monoclonal antibody against mouse TNFα and a TNFα-neutralizing TNFα receptor (TNFR) fusion molecule. From their studies Plessner et al conclude: ‘ . . . incomplete neutralization of TNF allows the host to maintain control of the infection.’

We believe, therefore, that the use of non-competitive TNFR1 antagonists to treat TNFR1-mediated diseases or conditions could be beneficial in that such positive effects of TNFα could be retained.

Neutralisation of TNFR1 can be determined in a cell assay, e.g. in a standard MRC5 assay as determined by inhibition of TNF alpha-induced IL-8 secretion. The assay is based on the induction of IL-8 secretion by TNFα in MRC-5 cells and is adapted from the method described in Akeson, A. et al. Journal of Biological Chemistry 271:30517-30523 (1996), describing the induction of IL-8 by IL-1 in HUVEC.

In some embodiments of the invention, the TNFR1 binding protein may be cross-reactive with TNFR1 in other species. Thus, neutralisation of mouse TNFR1 can be determined in a standard L929 assay as determined by inhibition of TNF alpha-induced cytotoxicity; or in a standard Cynomolgus KI assay as determined by inhibition of TNF alpha-induced IL-8 secretion. Details of standard assays for TNFR1 antagonists are known in the art, e.g. in WO2006038027, WO2008149144, WO2008149148 and WO20110066914. Accordingly, in an embodiment, the TNFR1 binding protein, at a concentration of 100 nM, inhibits human TNFR1 signaling by:
(i) >50% in a standard MRC5 cell assay in the presence of human TNFα at a TNFαconcentration in the assay of 100 pg/ml as determined by inhibition of IL-8 secretion using an immuno-sandwich method, and
(ii) ≦50% in a standard MRC5 cell assay in the presence of human TNFα at a TNFαconcentration in the assay of 2 ng/ml or more (e.g. 5 ng/ml) as determined by said immuno-sandwich method.

MRC-5 cells are available from ATCC and have been deposited under ATCC accession number CCL-171. In one embodiment, the MRC5 cell assays in (i) and (ii) are carried out at 37 degrees centigrade, each assay optionally for 18 hours. In one embodiment, in each assay the antagonist is pre-incubated with MRC5 cells (for example, for 60 minutes) prior to adding the TNFα. This pre-incubation time is not counted in the 18 hours assay time mentioned above. The TNFα can be from any source. The concentrations of TNFα used in assays herein can be determined by conventional techniques. In one embodiment, the TNFα is from Peprotech. The sequence of human TNFα is as follows:

(SEQ ID NO: 4)
VRSSSRTPSD KPVAHVVANP QAEGQLQWLN RRANALLANG
VELRDNQLVV PSEGLYLIYS QVLFKGQGCP STHVLLTHTI
SRIAVSYQTK VNLLSAIKSP CQRETPEGAE AKPWYEPIYL
GGVFQLEKGD RLSAEINRPD YLDFAESGQV YFGIIAL

Known immuno-sandwich methods can be used, and these will be evident to the skilled addressee. For example, the immuno-sandwich method is selected from ELISA, using a calorimetric detection, the Applied Biosystems 8200 cellular detection system (FMAT), using fluorescence detection and Meso Scale Discovery (MSD), using electrochemiluminescence detection.

In a particular embodiment, the assay is carried out as follows. The human fibroblast cell line MRC-5 was incubated with a dose-range of TNFR1 binding protein and then stimulated with 200 pg/ml of TNFα (Peprotech) for 18 h. After this stimulation, the media was removed and the levels of IL-8 in the media, produced by the cells in response to TNFα, was determined using the AB18200 (Applied Biosystems). The ability of the TNFR1 binding protein to block the secretion of IL-8 is a functional read-out of how well they inhibit TNFR1-mediated signaling.

In a still more specific embodiment, the assay is carried out as follows. MRC-5 cells (ATCC number: CCL-171) are plated in microtitre plates (5×103 cells/well) and the cells are pre-incubated for 1 hour with a dose-range of TNFR1 binding protein followed by addition of a fixed amount of human TNFα (200 pg/ml). Following overnight incubation (18 h at 37° C.), the culture supernatant is aspirated and IL-8 release was determined using an IL-8 ABI 8200 cellular detection assay (FMAT). The IL-8 FMAT assay used detection and capture reagents from R&D Systems. Beads, goat anti-mouse IgG (H&L) coated polystyrene particles 0.5% w/v 6-8 μm (Spherotech Inc, Cat#MP-60-5), were coated with the capture antibody mouse monoclonal anti-human IL-8 antibody (R&D systems, Cat# MAB208). For detection, biotinylated goat anti-human IL-8 antibody (R&D systems, Cat# BAF208) and Streptavidin Alexafluor 647 (Molecular Probes, Cat#S32357) are used. Recombinant human IL-8 (R&D systems, Cat#208-IL) was used as the standard.

In one embodiment, the TNFR1 binding protein antagonises both human and murine TNFR1. Functional mouse cross-reactivity can be determined using the mouse L929 cell line, in which the level of protection provided by the TNFR1 binding protein against TNFα-induced cytotoxicity is evaluated. In this assay, cells are again incubated with a dose-range of TNFR1 binding protein followed by stimulation with TNFα in the presence of actinomycine. After overnight incubation, the viability of the cells is measured and plotted against TNFR1 binding protein concentration.

In one embodiment, the TNFR1 binding protein antagonises both human and Cynomolgus monkey TNFR1. Cynomologous monkey cross-reactivity of the TNFR1 binding protein can be tested using the CYNOM-K1 cell line. Briefly, the TNFR1 binding protein is incubated with CYNOM-K1 cells (ECACC 90071809) (5×10³ cells/well) for one hour at 37° C. in a flat bottom cell culture plate. Recombinant human TNF alpha (Peprotech) is added (final concentration of 200 pg/ml) and the plates are incubated for 18-20 hours. The level of secreted IL-8 is then measured in the culture supernatant using the DuoSet ELISA development system (R&D Systems, cat# DY208), according to the manufacturer's instructions (document number 750364.16 version 11/08). The ND50 is determined by plotting TNFR1 binding protein concentration against the percentage of inhibition of IL-8 secretion.

Signalling through TNF receptor 1 (TNFR1, p55) can be inhibited either directly through competitive inhibition of TNFα binding to its receptor or indirectly by a non-competitive mechanism in which the binding of TNFα to its receptor is not affected by the presence of the inhibitor. To discriminate between these two classes of TNFR1-signaling inhibitors, the combined information from a cell-based, TNFα-induced, cytokine release assay (e.g. an MRC-5 assay as described above) and a receptor-binding assay can be used. Briefly, in one embodiment of a standard receptor binding assay, TNFR1 (e.g. TNFR1-Fc fusion (R&D Systems (Cat #372-RI), sequence is human TNFR1 (Leu30-Thr211 & Asp41-Thr211)-IEGRMD-Human IgG1 (Pro100-Lys330)-6 His-tag)) is coated on anti-IgG beads and incubated with a concentration range (e.g. 0.01 nM-10 μM) of a binding protein (e.g. a dAb) directed against TNFR1. Subsequently, TNFα is added followed by addition of a biotinylated anti-TNFα antibody and fluorescently-labeled streptavidin. The level of fluorescence for each measurement is determined (e.g. in an ABI 8200 cellular detection assay (FMAT)) and plotted against the corresponding TNFR1 binding protein concentration used. If the TNFR1 binding protein is competitive with TNFα binding to its receptor, the fluorescence will decrease with increasing concentrations of TNFR1 binding protein and consequently inhibition will be observed. Conversely, if the TNFR1 binding protein is non-competitive with TNFα binding to its receptor, the fluorescence will not change with increasing concentrations of TNFR1 binding protein and no inhibition will be observed. Hence, TNFR1 binding protein can be classified based on their ability to inhibit TNFαbinding to its receptor 1 in a standard RBA.

In an embodiment, the TNFR1 binding protein binds TNFRI and antagonizes the activity of the TNFR1 in a standard cell assay (e.g. an MRC5 assay as described herein) with an ND₅₀ of ≦100 nM, and at a concentration of 10 μM the dAb agonizes the activity of the TNFR1 by ≦5% in the assay.

In particular embodiments, the binding protein does not substantially agonize TNFR1 (act as an agonist of TNFR1) in a standard cell assay (i.e., when present at a concentration of 1 nM, 10 nM, 100 nM, 1 μM or 10 μM, results in no more than about 5% of the TNFR1-mediated activity induced by TNFα (100 pg/ml) in the assay).

In one embodiment, the TNFR1 binding protein of any aspect of the invention comprises or consists of an TNFR1 binding protein, e.g. a single variable domain, comprising a binding site that specifically binds:

(i) human TNFR1 with a dissociation constant (KD) of (or of about) 500 pM or less, 400 pM or less, 350 pM or less, 300 pM or less, 250 pM or less, 200 pM or less, or 150 pM or less as determined by surface plasmon resonance; and optionally also specifically binds

(ii) non-human primate TNFR1 (eg, Cynomolgus monkey, rhesus or baboon TNFR1) with a dissociation constant (KD) of (or of about) 500 pM or less, 400 pM or less, 350 pM or less, 300 pM or less, 250 pM or less, 200 pM or less, or 150 pM or less as determined by surface plasmon resonance; and/or

(iii) murine TNFR1 with a dissociation constant (KD) of (or of about) 7 nM or less, 6 nM or less, 5 nM or less, 4 nM or less, 3 nM or less, 2 nM or less, or 1 nM or less as determined by surface plasmon resonance.

In one embodiment, the TNFR1 binding protein of any aspect of the invention comprises or consists of a TNFR1 binding protein, e.g. a single variable domain, comprising a binding site that specifically binds

(i) human TNFR1 with an off-rate constant (Koff) of (or of about) 2×10⁻⁴ S⁻¹ or less, or 1×10⁻⁴ S⁻¹ or less, or 1×10⁻⁵ S⁻¹ or less as determined by surface plasmon resonance; and optionally also specifically binds

(ii) non-human primate TNFR1 (eg, Cynomolgus monkey, rhesus or baboon TNFR1) with an off-rate constant (Koff) of (or of about) 2×10⁻⁴ S⁻¹ or less, 1×10⁻⁴ S⁻¹ or less, or 1×10⁻⁵ S⁻¹ or less as determined by surface plasmon resonance; and/or

(iii) murine TNFR1 with an off-rate constant (Koff) of (or of about) 1×10⁻³ S⁻¹ or less, or 1×10⁻⁴ S⁻¹ or less as determined by surface plasmon resonance.

In one embodiment, the TNFR1 binding protein of any aspect of the invention comprises or consists of an TNFR1 binding protein, e.g. a single variable domain, comprising a binding site that specifically binds

(i) human TNFR1 with an on-rate constant (Kon) of (or of about) 5×10⁴ M⁻¹ s⁻¹ or more, 1×10⁵ M⁻¹ s⁻¹ or more, 2×10⁵ M⁻¹ s⁻¹ or more, 3×10⁵ M⁻¹ s⁻¹ or more, 4×10⁵ M⁻¹ s⁻¹ or more, or 5×10⁵ M⁻¹ s⁻¹ or more as determined by surface plasmon resonance; and optionally also specifically binds

(ii) non-human primate TNFR1 (eg, Cynomolgus monkey, rhesus or baboon TNFR1) with an on-rate constant (Kon) of (or of about) 5×10⁴ M⁻¹ s⁻¹ or more, 1×10⁵ M⁻¹ s⁻¹ or more, 2×10⁵ M⁻¹ s⁻¹ or more, 3×10⁵ M⁻¹ s⁻¹ or more, 4×10⁵ M⁻¹ s⁻¹ or more, or 5×10⁵ M⁻¹ s⁻¹ or more as determined by surface plasmon resonance; and/or

(iii) murine TNFR1 with an on-rate constant (Kon) of (or of about) 0.5×10⁵ M⁻¹ s⁻¹ or more, 1×10⁵ M⁻¹ s⁻¹ or more, or 2×10⁵ M⁻¹ s⁻¹ or more as determined by surface plasmon resonance.

In one embodiment, the TNFR1 binding protein of the invention comprises or consists of a single variable domain that specifically binds human, Cynomologus monkey and optionally canine TNFR1. Specific binding is indicated by a dissociation constant KD of 10 micromolar or less, optionally 1 micromolar or less. Specific binding of an antigen-binding protein to an antigen or epitope can be determined by a suitable assay, including, for example, Scatchard analysis and/or competitive binding assays, such as radioimmunoassays (RIA), enzyme immunoassays such as ELISA and sandwich competition assays, and the different variants thereof. In one example, the TNFR1 binding protein also specifically binds murine TNFR1.

In one embodiment of any aspect of the invention, the TNFR1 binding protein is an antagonist which neutralizes TNFR1 with an ND50 of (or about of) 5, 4, 3, 2 or 1 nM or less in a standard MRC5 assay as determined by inhibition of TNF alpha-induced IL-8 secretion.

In one embodiment of any aspect of the invention, the antagonist also neutralizes (murine) TNFR1 with an ND50 of 150, 100, 50, 40, 30 or 20 nM or less; or from (about) 150 to 10 nM; or from (about) 150 to 20 nM; or from (about) 110 to 10 nM; or from (about) 110 to 20 nM in a standard L929 assay as determined by inhibition of TNF alpha-induced cytotoxicity.

In one embodiment of any aspect of the invention, the antagonist also neutralizes (Cynomolgus monkey) TNFR1 with an ND50 of 5, 4, 3, 2 or 1 nM or less; or (about) 5 to (about) 1 nM in a standard Cynomologus KI assay as determined by inhibition of TNF alpha-induced IL-8 secretion.

The TNFR1 binding proteins of the present invention may be specific antagonists of TNFR1, in that they do not antagonize (inhibit signal transduction mediated through) TNFR2, and/or do not antagonize (inhibit signal transduction mediated through) other members of the TNF/NGF receptor superfamily.

The TNFR1 binding proteins of the present invention may be non-competitive antagonists of TNFR1, in that the TNFR1 binding protein binds to human TNFR1 (SEQ ID NO:1) but does not compete with or inhibit TNFα for binding to TNFR1 (e.g. in a standard receptor binding assay). In this embodiment, in one example the TNFR1 binding protein (e.g. an anti-TNFR1 immunoglobulin variable domain) specifically binds to an epitope consisting of residues within domains 1, 2, 3 or 4 of TNFR1. More particularly, the TNR1 binding protein binds to an epitope consisting of residues in domain 4, or in Domain 3.

Typically, the TNFR1 binding proteins according to the invention are monovalent and contain one binding site that interacts with TNFR1. Monovalent binding proteins bind one TNFR1 and may not induce cross-linking or clustering of TNFR1 on the surface of cells which can lead to activation of the receptor and signal transduction. They can therefore be useful antagonists of TNFR1. In an embodiment, the monovalent antagonist binds to an epitope which spans more than one Domain of TNFR1.

Multivalent TNFR1 binding proteins may also have a first binding site for TNFR1 and a second binding site for a separate antigen (for example human serum albumin). Multivalent TNFR1 binding proteins which are capable of binding TNFR1 and at least one different antigen may also be referred to herein as “multispecific ligands”.

As used herein, the term “prevention” and “preventing” involves administration of the protective composition prior to the induction of the disease or condition. “Treatment” and “treating” involves administration of the protective composition after disease or condition symptoms become manifest. “Suppression” or “suppressing” refers to administration of the composition after an inductive event, but prior to the clinical appearance of the disease or condition.

In certain embodiments, the TNFR1 binding proteins of the invention are efficacious in models of chronic inflammatory diseases when an effective amount is administered. Generally an effective amount is about 1 mg/kg to about 10 mg/kg (e.g., about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, or about 10 mg/kg). The models of chronic inflammatory disease (see those described in WO2006038027) are recognized by those skilled in the art as being predictive of therapeutic efficacy in humans.

In particular embodiments, the TNFR1 binding protein is efficacious in the standard mouse collagen-induced arthritis model (see WO2006038027 for details of the model). For example, administering an effective amount of the TNFR1 binding protein can reduce the average arthritic score of the summation of the four limbs in the standard mouse collagen-induced arthritis model, for example, by about 1 to about 16, about 3 to about 16, about 6 to about 16, about 9 to about 16, or about 12 to about 16, as compared to a suitable control. In another example, administering an effective amount of the TNFR1 binding protein can delay the onset of symptoms of arthritis in the standard mouse collagen-induced arthritis model, for example, by about 1 day, about 2 days, about 3 days, about 4 days, about 5 days, about 6 days, about 7 days, about 10 days, about 14 days, about 21 days or about 28 days, as compared to a suitable control. In another example, administering an effective amount of the TNFR1 binding protein can result in an average arthritic score of the summation of the four limbs in the standard mouse collagen-induced arthritis model of 0 to about 3, about 3 to about 5, about 5 to about 7, about 7 to about 15, about 9 to about 15, about 10 to about 15, about 12 to about 15, or about 14 to about 15.

In other embodiments, the TNFR1 binding protein is efficacious in the mouse ΔARE model of arthritis (see WO2006038027 for details of the model). For example, administering an effective amount of the TNFR1 binding protein can reduce the average arthritic score in the mouse ΔARE model of arthritis, for example, by about 0.1 to about 2.5, about 0.5 to about 2.5, about 1 to about 2.5, about 1.5 to about 2.5, or about 2 to about 2.5, as compared to a suitable control. In another example, administering an effective amount of the TNFR1 binding protein can delay the onset of symptoms of arthritis in the mouse ΔARE model of arthritis by, for example, about 1 day, about 2 days, about 3 days, about 4 days, about 5 days, about 6 days, about 7 days, about 10 days, about 14 days, about 21 days or about 28 days, as compared to a suitable control. In another example, administering an effective amount of the TNFR1 binding protein can result in an average arthritic score in the mouse ΔARE model of arthritis of 0 to about 0.5, about 0.5 to about 1, about 1 to about 1.5, about 1.5 to about 2, or about 2 to about 2.5.

In other embodiments, the TNFR1 binding protein is efficacious in the mouse ΔARE model of inflammatory bowel disease (IBD) (see WO2006038027 for details of the model). For example, administering an effective amount of the TNFR1 binding protein can reduce the average acute and/or chronic inflammation score in the mouse ΔARE model of IBD, for example, by about 0.1 to about 2.5, about 0.5 to about 2.5, about 1 to about 2.5, about 1.5 to about 2.5, or about 2 to about 2.5, as compared to a suitable control. In another example, administering an effective amount of the TNFR1 binding protein can delay the onset of symptoms of IBD in the mouse ΔARE model of IBD by, for example, about 1 day, about 2 days, about 3 days, about 4 days, about 5 days, about 6 days, about 7 days, about 10 days, about 14 days, about 21 days or about 28 days, as compared to a suitable control. In another example, administering an effective amount of the TNFR1 binding protein can result in an average acute and/or chronic inflammation score in the mouse ΔARE model of IBD of 0 to about 0.5, about 0.5 to about 1, about 1 to about 1.5, about 1.5 to about 2, or about 2 to about 2.5.

In other embodiments, the TNFR1 binding protein is efficacious in the mouse dextran sulfate sodium (DSS) induced model of IBD (see WO2006038027 for details of the model). For example, administering an effective amount of the TNFR1 binding protein can reduce the average severity score in the mouse DSS model of IBD, for example, by about 0.1 to about 2.5, about 0.5 to about 2.5, about 1 to about 2.5, about 1.5 to about 2.5, or about 2 to about 2.5, as compared to a suitable control. In another example, administering an effective amount of the TNFR1 binding protein can delay the onset of symptoms of IBD in the mouse DSS model of IBD by, for example, about 1 day, about 2 days, about 3 days, about 4 days, about 5 days, about 6 days, about 7 days, about 10 days, about 14 days, about 21 days or about 28 days, as compared to a suitable control. In another example, administering an effective amount of the TNFR1 binding protein can result in an average severity score in the mouse DSS model of IBD of 0 to about 0.5, about 0.5 to about 1, about 1 to about 1.5, about 1.5 to about 2, or about 2 to about 2.5.

In particular embodiments, the TNFR1 binding protein is efficacious in the mouse tobacco smoke model of chronic obstructive pulmonary disease (COPD) (see WO2006038027 and WO2007049017 for details of the model). For example, administering an effective amount of the TNFR1 binding protein can reduce or delay onset of the symptoms of COPD, as compared to a suitable control.

Animal model systems which can be used to screen the effectiveness of the antagonists of TNFR1 (e.g. binding proteins thereof) in protecting against or treating the disease are available. Methods for the testing of systemic lupus erythematosus (SLE) in susceptible mice are known in the art (Knight et al. (1978) J. Exp. Med., 147: 1653; Reinersten et al. (1978) New Eng. J. Med., 299: 515). Myasthenia Gravis (MG) is tested in SJL/J female mice by inducing the disease with soluble AchR protein from another species (Lindstrom et al. (1988) Adv. Immunol., 42: 233). Arthritis is induced in a susceptible strain of mice by injection of Type II collagen (Stuart et al. (1984) Ann. Rev. Immunol., 42: 233). A model by which adjuvant arthritis is induced in susceptible rats by injection of mycobacterial heat shock protein has been described (Van Eden et al. (1988) Nature, 331: 171). Thyroiditis is induced in mice by administration of thyroglobulin as described (Maron et al. (1980) J. Exp. Med., 152: 1115). Insulin dependent diabetes mellitus (IDDM) occurs naturally or can be induced in certain strains of mice such as those described by Kanasawa et al. (1984) Diabetologia, 27: 113. EAE in mouse and rat serves as a model for MS in human. In this model, the demyelinating disease is induced by administration of myelin basic protein (see Paterson (1986) Textbook of Immunopathology, Mischer et al., eds., Grune and Stratton, New York, pp. 179-213; McFarlin et al. (1973) Science, 179: 478: and Satoh et al. (1987) J. Immunol., 138: 179).

The invention provides the TNFR1 binding protein of any aspect for treating and/or prophylaxis of an inflammatory condition. The invention provides the use of the TNFR1 binding protein of any aspect in the manufacture of a medicament for treating and/or prophylaxis of an inflammatory condition. In one embodiment, the condition is selected from the group consisting of arthritis, multiple sclerosis, inflammatory bowel disease and chronic obstructive pulmonary disease. In one example, the arthritis is rheumatoid arthritis or juvenile rheumatoid arthritis. In one example, the inflammatory bowel disease is selected from the group consisting of Crohn's disease and ulcerative colitis. In one example, the chronic obstructive pulmonary disease is selected from the group consisting of chronic bronchitis, chronic obstructive bronchitis and emphysema. In one example, the pneumonia is bacterial pneumonia. In one example, the bacterial pneumonia is Staphylococcal pneumonia.

The invention also provides a TNFR1 binding protein of any aspect for treating and/or prophylaxis of a respiratory disease. The invention provides the use of the TNFR1 binding protein of any aspect in the manufacture of a medicament for treating and/or prophylaxis of a respiratory disease. In one example the respiratory disease is selected from the group consisting of lung inflammation, chronic obstructive pulmonary disease, asthma, pneumonia, hypersensitivity pneumonitis, pulmonary infiltrate with eosinophilia, environmental lung disease, pneumonia, bronchiectasis, cystic fibrosis, interstitial lung disease, primary pulmonary hypertension, pulmonary thromboembolism, disorders of the pleura, disorders of the mediastinum, disorders of the diaphragm, hypoventilation, hyperventilation, sleep apnea, acute respiratory distress syndrome, mesothelioma, sarcoma, graft rejection, graft versus host disease, lung cancer, allergic rhinitis, allergy, asbestosis, aspergilloma, aspergillosis, bronchiectasis, chronic bronchitis, emphysema, eosinophilic pneumonia, idiopathic pulmonary fibrosis, invasive pneumococcal disease, influenza, nontuberculous mycobacteria, pleural effusion, pneumoconiosis, pneumocytosis, pneumonia, pulmonary actinomycosis, pulmonary alveolar proteinosis, pulmonary anthrax, pulmonary edema, pulmonary embolus, pulmonary inflammation, pulmonary histiocytosis X, pulmonary hypertension, pulmonary nocardiosis, pulmonary tuberculosis, pulmonary veno-occlusive disease, rheumatoid lung disease, sarcoidosis, and Wegener's granulomatosis.

As used herein, the term “dose” refers to the quantity of TNFR1 binding protein administered to a subject all at one time (unit dose), or in two or more administrations over a defined time interval. For example, dose can refer to the quantity of TNFR1 binding protein administered to a subject over the course of one day (24 hours) (daily dose), two days, one week, two weeks, three weeks or one or more months (e.g., by a single administration, or by two or more administrations). The interval between doses can be any desired amount of time.

A “patient” is any animal, e.g., a mammal, e.g., a non-human primate (such as a baboon, rhesus monkey or Cynomolgus monkey), mouse, human, rabbit, rat, dog, cat or pig. In one embodiment, the patient is a human.

Generally, the present TNFR1 binding proteins will be utilised in purified form together with pharmacologically appropriate carriers.

The TNFR1 binding proteins of the present invention may be used as separately administered compositions or in conjunction with other agents. These can include various immunotherapeutic drugs, such as cylcosporine, methotrexate, adriamycin or cisplatinum, and immunotoxins. Pharmaceutical compositions can include “cocktails” of various cytotoxic or other agents in conjunction with the ligands of the present invention, or even combinations of ligands according to the present invention having different specificities, such as ligands selected using different target antigens or epitopes, whether or not they are pooled prior to administration.

The route of administration of pharmaceutical compositions according to the invention may be any of those commonly known to those of ordinary skill in the art. For therapy, including without limitation immunotherapy, the selected ligands thereof of the invention can be administered to any patient in accordance with standard techniques.

The administration can be by any appropriate mode, including parenterally, intravenously, intramuscularly, intraperitoneally, subcutaneously, transdermally, via the pulmonary route, or also, appropriately, by direct infusion with a catheter. The dosage and frequency of administration will depend on the age, sex and condition of the patient, concurrent administration of other drugs, counterindications and other parameters to be taken into account by the clinician. Administration can be local (e.g., local delivery to the lung by pulmonary administration, e.g., intranasal administration) or systemic as indicated.

The TNFR1 binding proteins of the invention can be lyophilised for storage and reconstituted in a suitable carrier prior to use.

The compositions containing the present TNFR1 binding proteins can be administered for prophylactic and/or therapeutic treatments. In certain therapeutic applications, an adequate amount to accomplish at least partial inhibition, suppression, modulation, killing, or some other measurable parameter, of a population of selected cells is defined as a “therapeutically-effective dose”. Amounts needed to achieve this dosage will depend upon the severity of the disease and the general state of the patient's own immune system, but generally range from 0.005 to 50.0 mg/kg of TNFR1 binding protein, e.g. dAb or antagonist per kilogram of body weight, with doses of 0.05 to 10.0 mg/kg/dose being more commonly used. For prophylactic applications, compositions containing the present TNFR1 binding proteins may also be administered in similar or slightly lower dosages, to prevent, inhibit or delay onset of disease (e.g., to sustain remission or quiescence, or to prevent acute phase). The skilled clinician will be able to determine the appropriate dosing interval to treat, suppress or prevent disease. When a TNFR1 binding protein is administered to treat, suppress or prevent a chronic inflammatory disease, it can be administered up to four times per day, twice weekly, once weekly, once every two weeks, once a month, or once every two months, at a dose off, for example, about 10 μg/kg to about 80 mg/kg, about 100 μg/kg to about 80 mg/kg, about 1 mg/kg to about 80 mg/kg, about 1 mg/kg to about 70 mg/kg, about 1 mg/kg to about 60 mg/kg, about 1 mg/kg to about 50 mg/kg, about 1 mg/kg to about 40 mg/kg, about 1 mg/kg to about 30 mg/kg, about 1 mg/kg to about 20 mg/kg, about 1 mg/kg to about 10 mg/kg, about 10 μg/kg to about 10 mg/kg, about 10 μg/kg to about 5 mg/kg, about 10 μg/kg to about 2.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg or about 10 mg/kg. In particular embodiments, TNFR1 binding protein, ligand or antagonist is administered to treat, suppress or prevent a chronic inflammatory disease once every two weeks or once a month at a dose of about 10 μg/kg to about 10 mg/kg (e.g., about 10 μg/kg, about 100 μg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg or about 10 mg/kg.)

Treatment or therapy performed using the TNFR1 binding proteins or compositions described herein is considered “effective” if one or more symptoms are reduced (e.g., by at least 10% or at least one point on a clinical assessment scale), relative to such symptoms present before treatment, or relative to such symptoms in an individual (human or model animal) not treated with such composition or other suitable control. Symptoms will obviously vary depending upon the disease or disorder targeted, but can be measured by an ordinarily skilled clinician or technician. Such symptoms can be measured, for example, by monitoring the level of one or more biochemical indicators of the disease or disorder (e.g., levels of an enzyme or metabolite correlated with the disease, affected cell numbers, etc.), by monitoring physical manifestations (e.g., inflammation, tumor size, etc.), or by an accepted clinical assessment scale, for example, the Expanded Disability Status Scale (for multiple sclerosis), the Irvine Inflammatory Bowel Disease Questionnaire (32 point assessment evaluates quality of life with respect to bowel function, systemic symptoms, social function and emotional status—score ranges from 32 to 224, with higher scores indicating a better quality of life), the Quality of Life Rheumatoid Arthritis Scale, or other accepted clinical assessment scale as known in the field. A sustained (e.g., one day or more, or longer) reduction in disease or disorder symptoms by at least 10% or by one or more points on a given clinical scale is indicative of “effective” treatment. Similarly, prophylaxis performed using a composition as described herein is “effective” if the onset or severity of one or more symptoms is delayed, reduced or abolished relative to such symptoms in a similar individual (human or animal model) not treated with the composition.

A pharmaceutical composition according to the present invention may be utilised in prophylactic and therapeutic settings to aid in the alteration, inactivation, killing or removal of a select target cell population in a mammal.

The TNFR1 binding proteins can be administered and or formulated together with one or more additional therapeutic or active agents. When a TNFR1 binding protein (e.g. a dAb) is administered with an additional therapeutic agent, the TNFR1 binding protein can be administered before, simultaneously with or subsequent to administration of the additional agent. Generally, the TNFR1 binding protein and additional agent are administered in a manner that provides an overlap of therapeutic effect.

In another aspect, the invention provides a method for treating, suppressing or preventing a chronic inflammatory disease, comprising administering to a mammal in need thereof a therapeutically-effective dose or amount of a TNFR1 binding protein according to the invention.

In another aspect, the invention provides a method for treating, suppressing or preventing arthritis (e.g., rheumatoid arthritis, juvenile rheumatoid arthritis, ankylosing spondylitis, psoriatic arthritis) comprising administering to a mammal in need thereof a therapeutically-effective dose or amount of a TNFR1 binding protein according to the invention.

In another aspect, the invention provides a method for treating, suppressing or preventing psoriasis comprising administering to a mammal in need thereof a therapeutically-effective dose or amount of a TNFR1 binding protein according to the invention.

In another aspect, the invention provides a method for treating, suppressing or preventing inflammatory bowel disease (e.g., Crohn's disease, ulcerative colitis) comprising administering to a mammal in need thereof a therapeutically-effective dose or amount of a TNFR1 binding protein according to the invention.

In another aspect, the invention provides a method for treating, suppressing or preventing chronic obstructive pulmonary disease (e.g., chronic bronchitis, chronic obstructive bronchitis, emphysema), comprising administering to a mammal in need thereof a therapeutically-effective dose or amount of a TNFR1 binding protein according to the invention.

In another aspect, the invention provides a method for treating, suppressing or preventing pneumonia (e.g., bacterial pneumonia, such as Staphylococcal pneumonia) comprising administering to a mammal in need thereof a therapeutically-effective dose or amount of a TNFR1 binding protein according to the invention.

The invention provides a method for treating, suppressing or preventing other pulmonary diseases in addition to chronic obstructive pulmonary disease, and pneumonia. Other pulmonary diseases that can be treated, suppressed or prevented in accordance with the invention include, for example, cystic fibrosis and asthma (e.g., steroid resistant asthma). Thus, in another embodiment, the invention is a method for treating, suppressing or preventing a pulmonary disease (e.g., cystic fibrosis, asthma) comprising administering to a mammal in need thereof a therapeutically-effective dose or amount of a TNFR1 binding protein according to the invention.

In particular embodiments, an antagonist of TNFR1 (a TNFR1 binding protein of the invention) is administered via pulmonary delivery, such as by inhalation (e.g., intrabronchial, intranasal or oral inhalation, intranasal drops) or by systemic delivery (e.g., parenteral, intravenous, intramuscular, intraperitoneal, subcutaneous).

In another aspect, the invention provides a method treating, suppressing or preventing septic shock comprising administering to a mammal in need thereof a therapeutically-effective dose or amount of a TNFR1 binding protein according to the invention.

In a further aspect of the invention, there is provided a composition comprising a TNFR1 binding protein according to the invention and a pharmaceutically acceptable carrier, diluent or excipient.

Moreover, the present invention provides a method for the treatment of disease using a TNFR1 binding protein, ligand or antagonist of TNFR1 or a composition according to the present invention. In an embodiment the disease is cancer or an inflammatory disease, e.g. rheumatoid arthritis, asthma or Crohn's disease.

In a further aspect of the invention, there is provided a composition comprising a TNFR1 binding protein, ligand or antagonist according to the invention and a pharmaceutically acceptable carrier, diluent or excipient.

In particular embodiments, the TNFR1 binding protein is administered via pulmonary delivery, such as by inhalation (e.g. intrabronchial, intranasal or oral inhalation, intranasal drops) or by systemic delivery (e.g. parenteral, intravenous, intramuscular, intraperitoneal, subcutaneous).

An aspect of the invention provides a pulmonary delivery device containing a TNFR1 binding protein or composition according to the invention. The device can be an inhaler or an intranasal administration device.

In some embodiments, any of the TNFR1 binding proteins described herein (e.g. a single variable domain) further comprises a half-life extending moiety, such as a polyalkylene glycol moiety, serum albumin or a fragment thereof, transferrin receptor or a transferrin-binding portion thereof, or a moiety comprising a binding site for a polypeptide that enhances half-life in vivo. In some embodiments, the half-life extending moiety is a moiety comprising a binding site for a polypeptide that enhances half-life in vivo selected from the group consisting of an affibody, a SpA domain, an LDL receptor class A domain, an EGF domain, and an avimer.

In other embodiments, the half-life extending moiety is a polyethylene glycol moiety. In one embodiment, the TNFR1 binding protein comprises (optionally consists of) a single variable domain of the invention linked to a polyethylene glycol moiety (optionally, wherein the moiety has a size of about 20 to about 50 kDa, optionally about 40 kDa linear or branched PEG). Reference is made to WO04081026 for more detail on PEGylation of dAbs and binding moieties. In one embodiment, the antagonist consists of a dAb monomer linked to a PEG, wherein the dAb monomer is a single variable domain according to the invention. This TNFR1 binding protein can be provided for treatment of inflammatory disease, a lung condition (e.g., asthma, influenza or COPD) or cancer or optionally is for intravenous administration.

In other embodiments, the half-life extending moiety is an antibody or antibody fragment (e.g. a single variable domain) comprising a binding site for serum albumin or neonatal Fc receptor.

In another aspect, the invention provides a multispecific binding protein, comprising a TNFR1 binding protein of the invention and a antibody or antibody fragment comprising a binding site for serum albumin or neonatal Fc receptor.

The invention also relates to a composition (e.g. a pharmaceutical composition) comprising a TNFR1 binding protein of the invention (e.g. a single variable domain) and a physiologically acceptable carrier. In some embodiments, the composition comprises a vehicle for intravenous, intramuscular, intraperitoneal, intraarterial, intrathecal, intraarticular, subcutaneous administration, pulmonary, intranasal, vaginal, or rectal administration.

The invention also relates to a drug delivery device comprising the composition (e.g. pharmaceutical composition) of the invention. In some embodiments, the drug delivery device comprises a plurality of therapeutically effective doses of ligand.

In other embodiments, the drug delivery device is selected from the group consisting of parenteral delivery device, intravenous delivery device, intramuscular delivery device, intraperitoneal delivery device, transdermal delivery device, pulmonary delivery device, intraarterial delivery device, intrathecal delivery device, intraarticular delivery device, subcutaneous delivery device, intranasal delivery device, vaginal delivery device, rectal delivery device, syringe, a transdermal delivery device, a capsule, a tablet, a nebulizer, an inhaler, an atomizer, an aerosolizer, a mister, a dry powder inhaler, a metered dose inhaler, a metered dose sprayer, a metered dose mister, a metered dose atomizer, and a catheter.

The TNFR1 binding protein (e.g. single variable domain or multispecific ligand containing a single variable domain) of the invention can be formatted as described herein. For example, the binding protein of the invention can be formatted to tailor in vivo serum half-life. If desired, the binding protein can further comprise a toxin or a toxin moiety as described herein. In some embodiments, the TNFR1 binding protein comprises a surface active toxin, such as a free radical generator (e.g. selenium containing toxin) or a radionuclide. In other embodiments, the toxin or toxin moiety is a polypeptide domain (e.g. a dAb) having a binding site with binding specificity for an intracellular target. In particular embodiments, the binding protein is an IgG-like format that has binding specificity for TNFR1 (e.g. human TNFR1).

Increased half-life is useful in in vivo applications of immunoglobulins, especially antibodies and most especially antibody fragments of small size. Such fragments (Fvs, disulphide bonded Fvs, Fabs, scFvs, dAbs) suffer from rapid clearance from the body; thus, whilst they are able to reach most parts of the body rapidly, and are quick to produce and easier to handle, their in vivo applications have been limited by their only brief persistence in vivo. One embodiment of the invention solves this problem by providing increased half-life of the TNFR1 binding proteins in vivo and consequently longer persistence times in the body of the functional activity of the TNFR1 binding proteins.

Methods for pharmacokinetic analysis and determination of binding protein half-life will be familiar to those skilled in the art. Details may be found in Kenneth, A et al: Chemical Stability of Pharmaceuticals: A Handbook for Pharmacists and in Peters et al, Pharmacokinetic analysis: A Practical Approach (1996). Reference is also made to “Pharmacokinetics”, M Gibaldi & D Perron, published by Marcel Dekker, 2^{nd} Rev. ex edition (1982), which describes pharmacokinetic parameters such as t alpha and t beta half lives and area under the curve (AUC). Half-life and AUC definitions are provided above.

In one embodiment, the present invention provides a TNFR1 binding protein according to the invention having a tα half-life in the range of 15 minutes or more. In one embodiment, the lower end of the range is 30 minutes, 45 minutes, 1 hour, 2 hours, 3 hours, 4 hours, 5 hours, 6 hours, 7 hours, 10 hours, 11 hours or 12 hours. In addition, or alternatively, a ligand or composition according to the invention will have a tα half life in the range of up to and including 12 hours. In one embodiment, the upper end of the range is 11, 10, 9, 8, 7, 6 or 5 hours. An example of a suitable range is 1 to 6 hours, 2 to 5 hours or 3 to 4 hours.

In one embodiment, the present invention provides a TNFR1 binding protein according to the invention having a tβ half-life in the range of about 2.5 hours or more. In one embodiment, the lower end of the range is about 3 hours, about 4 hours, about 5 hours, about 6 hours, about 7 hours, about 10 hours, about 11 hours, or about 12 hours. In addition, or alternatively, a ligand or composition according to the invention has a tβ half-life in the range of up to and including 21 days. In one embodiment, the upper end of the range is about 12 hours, about 24 hours, about 2 days, about 3 days, about 5 days, about 10 days, about 15 days or about 20 days. In one embodiment a ligand or composition according to the invention will have a tβ half life in the range about 12 to about 240 hours or 12 to 60 hours. In a further embodiment, it will be in the range about 12 to about 48 hours. In a further embodiment still, it will be in the range about 12 to about 26 hours.

In addition, or alternatively to the above criteria, the present invention provides a TNFR1 binding protein according to the invention having an AUC value (area under the curve) in the range of about 1 mg·min/ml or more. In one embodiment, the lower end of the range is about 5, about 10, about 15, about 20, about 30, about 100, about 200 or about 300 mg·min/ml. In addition, or alternatively, a ligand or composition according to the invention has an AUC in the range of up to about 600 mg·min/ml. In one embodiment, the upper end of the range is about 500, about 400, about 300, about 200, about 150, about 100, about 75 or about 50 mg·min/ml. In one embodiment a ligand according to the invention will have a AUC in the range selected from the group consisting of the following: about 15 to about 150 mg·min/ml, about 15 to about 100 mg·min/ml, about 15 to about 75 mg·min/ml, and about 15 to about 50 mg·min/ml.

TNFR1 binding proteins of the invention can be formatted to have a larger hydrodynamic size, for example, by attachment of a PEG group, serum albumin, transferrin, transferrin receptor or at least the transferrin-binding portion thereof, an antibody Fc region, or by conjugation to an antibody domain. For example, polypeptides dAbs and antagonists formatted as a larger antigen-binding fragment of an antibody or as an antibody (e.g. formatted as a Fab, Fab′, F(ab)₂, F(ab′)₂, IgG, scFv).

Hydrodynamic size of the TNFR1 binding proteins of the invention may be determined using methods which are well known in the art. For example, gel filtration chromatography may be used to determine the hydrodynamic size of a TNFR1 binding protein. Suitable gel filtration matrices for determining the hydrodynamic sizes of proteins, such as cross-linked agarose matrices, are well known and readily available.

The size of a binding protein format (e.g. the size of a PEG moiety attached to a dAb monomer), can be varied depending on the desired application. For example, where binding protein is intended to leave the circulation and enter into peripheral tissues, it is desirable to keep the hydrodynamic size of the binding protein low to facilitate extravazation from the blood stream. Alternatively, where it is desired to have the binding protein remain in the systemic circulation for a longer period of time the size of the binding protein can be increased, for example by formatting as an Ig like protein.

The hydrodynamic size of a TNFR1 binding protein and its serum half-life can also be increased by conjugating or associating an TNFR1 binding polypeptide of the invention to a binding domain (e.g. antibody or antibody fragment that has the capability of specifically binding an antigen) that binds an antigen or epitope that increases half-live in vivo, as described herein. For example, the TNFR1 binding protein can be conjugated or linked to an anti-serum albumin or anti-neonatal Fc receptor antibody or antibody fragment, e.g. an anti-SA or anti-neonatal Fc receptor dAb, Fab, Fab′ or scFv, or to an anti-SA affibody or anti-neonatal Fc receptor Affibody or an anti-SA avimer, or an anti-SA binding domain which comprises a scaffold selected from, but not limited to, the group consisting of CTLA-4, lipocallin, SpA, an affibody, an avimer, GroEl and fibronectin (see WO2008096158 for disclosure of these binding domains, which domains and their sequences are incorporated herein by reference and form part of the disclosure of the present text). Conjugating refers to a composition comprising TNFR1 binding protein of the invention that is bonded (covalently or noncovalently) to a binding domain that binds serum albumin. In related embodiments, multispecific binding proteins according to the invention can be provided by bonding (covalently or noncovalently) the TNFR1 binding protein to a binding domain that binds to another antigen, for example a non-TNFR1 antigen (or another or the same epitope on TNFR1).

Suitable polypeptides that enhance serum half-life in vivo include, for example, transferrin receptor specific ligand-neuropharmaceutical agent fusion proteins (see U.S. Pat. No. 5,977,307, the teachings of which are incorporated herein by reference), brain capillary endothelial cell receptor, transferrin, transferrin receptor (e.g. soluble transferrin receptor), insulin, insulin-like growth factor 1 (IGF 1) receptor, insulin-like growth factor 2 (IGF 2) receptor, insulin receptor, blood coagulation factor X, al-antitrypsin and HNF 1a. Suitable polypeptides that enhance serum half-life also include alpha-1 glycoprotein (orosomucoid; AAG), alpha-1 antichymotrypsin (ACT), alpha-1 microglobulin (protein HC; AIM), antithrombin III (AT III), apolipoprotein A-1 (Apo A-1), apolipoprotein B (Apo B), ceruloplasmin (Cp), complement component C3 (C3), complement component C4 (C4), C1 esterase inhibitor (C1 INH), C-reactive protein (CRP), ferritin (FER), hemopexin (HPX), lipoprotein(a) (Lp(a)), mannose-binding protein (MBP), myoglobin (Myo), prealbumin (transthyretin; PAL), retinol-binding protein (RBP), and rheumatoid factor (RF).

Suitable proteins from the extracellular matrix include, for example, collagens, laminins, integrins and fibronectin. Collagens are the major proteins of the extracellular matrix. About 15 types of collagen molecules are currently known, found in different parts of the body, e.g, type I collagen (accounting for 90% of body collagen) found in bone, skin, tendon, ligaments, cornea, internal organs or type II collagen found in cartilage, vertebral disc, notochord, and vitreous humor of the eye.

Suitable proteins from the blood include, for example, plasma proteins (e.g, fibrin, α-2 macroglobulin, serum albumin, fibrinogen (e.g, fibrinogen A, fibrinogen B), serum amyloid protein A, haptoglobin, profilin, ubiquitin, uteroglobulin and β-2-microglobulin), enzymes and enzyme inhibitors (e.g, plasminogen, lysozyme, cystatin C, alpha-1-antitrypsin and pancreatic trypsin inhibitor), proteins of the immune system, such as immunoglobulin proteins (e.g, IgA, IgD, IgE, IgG, IgM, immunoglobulin light chains (kappa/lambda)), transport proteins (e.g, retinol binding protein, α-1 microglobulin), defensins (e.g, beta-defensin 1, neutrophil defensin 1, neutrophil defensin 2 and neutrophil defensin 3) and the like.

Suitable proteins found at the blood brain barrier or in neural tissue include, for example, melanocortin receptor, myelin, ascorbate transporter and the like.

Suitable polypeptides that enhance serum half-life in vivo also include proteins localized to the kidney (e.g, polycystin, type IV collagen, organic anion transporter KI, Heymann's antigen), proteins localized to the liver (e.g, alcohol dehydrogenase, G250), proteins localized to the lung (e.g, secretory component, which binds IgA), proteins localized to the heart (e.g, HSP 27, which is associated with dilated cardiomyopathy), proteins localized to the skin (e.g, keratin), bone specific proteins such as morphogenic proteins (BMPs), which are a subset of the transforming growth factor β superfamily of proteins that demonstrate osteogenic activity (e.g, BMP-2, BMP-4, BMP-5, BMP-6, BMP-7, BMP-8), tumor specific proteins (e.g, trophoblast antigen, herceptin receptor, oestrogen receptor, cathepsins (e.g, cathepsin B, which can be found in liver and spleen)).

Suitable disease-specific proteins include, for example, antigens expressed only on activated T-cells, including LAG-3 (lymphocyte activation gene), osteoprotegerin ligand (OPGL; see Nature 402, 304-309 (1999)), OX40 (a member of the TNF receptor family, expressed on activated T cells and specifically up-regulated in human T cell leukemia virus type-I (HTLV-I)-producing cells; see Immunol. 165 (1):263-70 (2000)). Suitable disease-specific proteins also include, for example, metalloproteases (associated with arthritis/cancers) including CG6512 Drosophila, human paraplegin, human FtsH, human AFG3L2, murine ftsH; and angiogenic growth factors, including acidic fibroblast growth factor (FGF-1), basic fibroblast growth factor (FGF-2), vascular endothelial growth factor/vascular permeability factor (VEGF/VPF), transforming growth factor-α (TGF α), tumor necrosis factor-alpha (TNF-α), angiogenin, interleukin-3 (IL-3), interleukin-8 (IL-8), platelet-derived endothelial growth factor (PD-ECGF), placental growth factor (P1GF), midkine platelet-derived growth factor-BB (PDGF), and fractalkine.

Suitable polypeptides that enhance serum half-life in vivo also include stress proteins such as heat shock proteins (HSPs). HSPs are normally found intracellularly. When they are found extracellularly, it is an indicator that a cell has died and spilled out its contents. This unprogrammed cell death (necrosis) occurs when as a result of trauma, disease or injury, extracellular HSPs trigger a response from the immune system. Binding to extracellular HSP can result in localizing the compositions of the invention to a disease site.

Suitable proteins involved in Fc transport include, for example, Brambell receptor (also known as FcRB). This Fc receptor has two functions, both of which are potentially useful for delivery. The functions are (1) transport of IgG from mother to child across the placenta (2) protection of IgG from degradation thereby prolonging its serum half-life. It is thought that the receptor recycles IgG from endosomes. (See, Holliger et al, Nat Biotechnol 15(7):632-6 (1997).)

The invention in one embodiment provides a TNFR1 binding protein and a second binding protein that binds serum albumin (SA). For example, the invention provides a dual specific binding protein comprising an anti-TNFR1 dAb (a first dAb) and an anti-SA dAb (a second dAb). The second binding protein (e.g. the second dAb) may bind SA with a KD as determined by surface plasmon resonance of about 1 nM to about 1, about 2, about 3, about 4, about 5, about 10, about 20, about 30, about 40, about 50, about 60, about 70, about 100, about 200, about 300, about 400 or about 500 μM (i.e., ×10⁻⁹ to 5×10⁻⁴M), or about 100 nM to about 10 μM, or about 1 to about 5 μM or about 3 to about 70 nM or about 10 nM to about 1, about 2, about 3, about 4 or about 5 μM. For example about 30 to about 70 nM as determined by surface plasmon resonance. In one embodiment, the anti-SA binding protein binds SA (e.g., HSA) with a KD as determined by surface plasmon resonance of approximately about 1, about 50, about 70, about 100, about 150, about 200, about 300 nM or about 1, about 2 or about 3 μM. In one embodiment, for a dual specific binding protein comprising a first anti-SA dAb and a second dAb to TNFR1, the affinity (e.g., KD and/or K_{off} as measured by surface plasmon resonance, e.g., using BiaCore) of the second dAb for its target is from about 1 to about 100000 times (e.g. about 100 to about 100000, or about 1000 to about 100000, or about 10000 to about 100000 times) the affinity of the first dAb for SA. In one embodiment, the serum albumin is human serum albumin (HSA). For example, the first dAb binds SA with an affinity of approximately about 10 μM, while the second dAb binds its target with an affinity of about 100 μM. In one embodiment, the serum albumin is human serum albumin (HSA). In one embodiment, the first dAb binds SA (e.g., HSA) with a KD of approximately about 50, for example about 70, about 100, about 150 or about 200 nM. Details of dual specific ligands are found in WO03002609, WO04003019, WO2008096158 and WO04058821.

In an aspect, the invention provides a fusion protein comprising the TNFR1 binding protein of the invention. The TNFR1 binding protein (e.g. a variable domain) can be fused, for example, to a peptide or polypeptide or protein. In one embodiment, the TNFR1 binding protein is fused to an antibody or antibody fragment, e.g. a monoclonal antibody or an Fc domain. Generally, fusion can be achieved by expressing the fusion product from a single nucleic acid sequence or by expressing a polypeptide comprising the TNFR1 binding protein and then assembling this polypeptide into a larger protein or antibody format using techniques that are conventional.

In one embodiment, the TNFR1 binding protein (e.g. the immunoglobulin single variable domain), antagonist or the ligand comprises an antibody constant domain. In one embodiment, the immunoglobulin single variable domain, antagonist or the fusion protein comprises an antibody Fc, optionally wherein the N-terminus of the Fc is linked (optionally directly linked) to the C-terminus of the variable domain.

Reference is made to WO2006038027, which discloses anti-TNFR1 immunoglobulin single variable domains. The disclosure of this document is incorporated herein in its entirety, in particular to provide for uses, formats, methods of selection, methods of production, methods of formulation and assays for anti-TNFR1 single variable domains, ligands, antagonists and the like, so that these disclosures can be applied specifically and explicitly in the context of the present invention, including to provide explicit description for importation into claims of the present disclosure.

In one embodiment of any aspect of the invention, the antagonist comprises or consists of a TNFR1 binding protein (e.g. a single variable domain) which comprises a terminal, optionally C-terminal, cysteine residue. For example, the cysteine residue can be used to attach PEG to the variable domain, eg, using a maleimide linkage (see, e.g. WO04081026).

### EXAMPLES

The present inventors have, for the first time, elucidated the crystallographic structure of the TNFR1-TNFα complex, thereby identifying those specific residues which are involved in receptor-ligand complexation. More importantly, the inventors have determined the significance and therapeutic potential of preventing dimerisation of TNFR1, as exemplified by the use of a domain antibody which binds to certain residues in the dimer interface and thereby blocks dimerisation, without competing with TNFα for binding to the receptor.

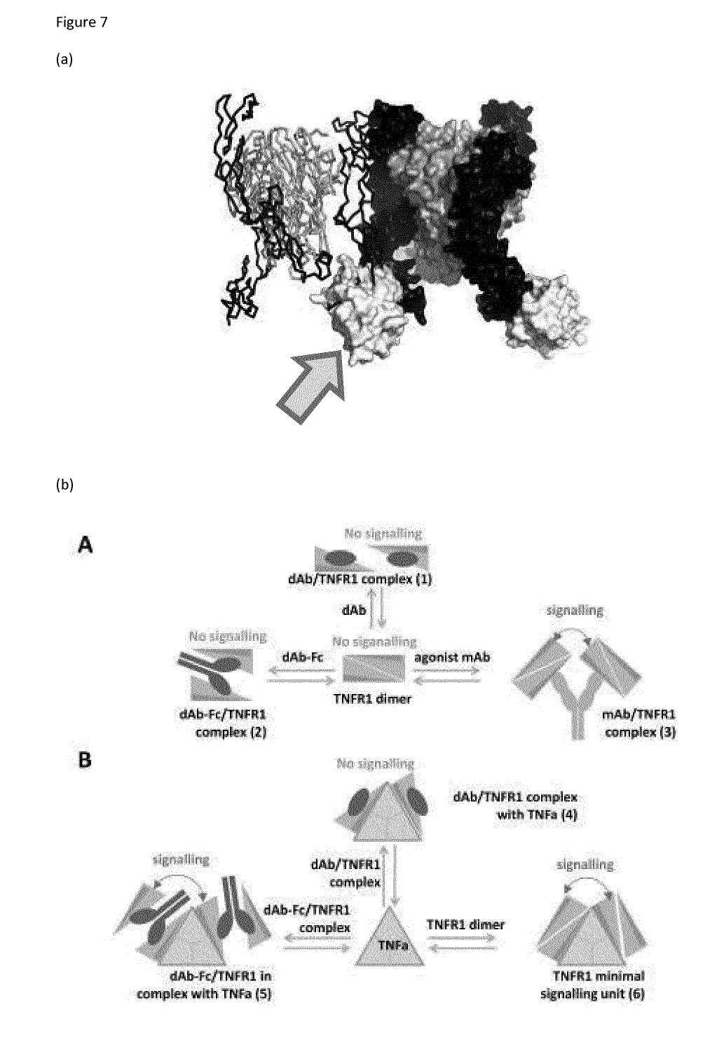

Preventing dimerisation of TNFR1, by binding to the residues involved in TNFR1 dimerisation, is shown to prevent TNF-a-mediated signalling through inhibition of formation of a minimal signalling unit (FIG. 7b) as well as subsequent signal amplification by TNFR1-TNFα clusters. Incomplete receptor occupancy, high local TNF-α concentrations, or higher-order cluster formation might still lead to weak signalling.

This could enable the development of a new class of TNF pathway inhibitors for diseases in which TNFα is present at pathogenic levels. This new class of inhibitor can reduce the potential for adverse reaction to TNFα inhibitors or competitive TNFR1 inhibitors, by allowing beneficial residual TNFα signalling while inhibiting the pathogenic effects of excess TNFα. TNFR1 binding proteins which bind to the residues identified herein as being involved in the TNFR1 dimerisation interface, in particular those residues in Domains 3 and 4, are expected to share the beneficial properties of DOM1h-574-208.

### Example 1

### Functional Characterisation of DOM1h-574-208

Signalling through TNF receptor 1 (TNFR1, p55) can be inhibited either directly through competitive inhibition of TNFα binding to its receptor or indirectly by a non-competitive mechanism in which the binding of TNFα to its receptor is not affected by the presence of the inhibitor.

To discriminate between these two classes of TNFR1-signaling inhibitors, the combined information from a receptor-binding assay and a cell-based, TNFα-induced, functional assay can be used. Suitable assays are described in WO2011051217.

Briefly, in the standard receptor binding assay TNFR1-Fc fusion (R&D Systems (Cat #372-RI), sequence is human TNFR1 (Leu30-Thr211 & Asp41-Thr211)-IEGRMD-Human IgG1 (Pro100-Lys330)-6 His-tag) is coated on anti-IgG beads and incubated with a concentration range (e.g. 0.01 nM-10 μM) of a domain antibody directed against TNFR1. Subsequently, TNFα is added followed by addition of a biotinylated anti-TNFα antibody and fluorescently-labelled streptavidin. The level of fluorescence for each measurement is determined in an ABI 8200 cellular detection assay (FMAT) and plotted against the corresponding dAb concentration used. A similar method can be used for antagonists and inhibitors of TNFR1 other than dAbs. If the anti-TNFR1 dAb is competitive with TNFα binding to its receptor, the fluorescence will decrease with increasing concentrations of dAb and consequently inhibition will be observed. Conversely, if the anti-TNFR1 dAb is non-competitive with TNFα binding to its receptor, the fluorescence will not change with increasing concentrations of dAb and no inhibition will be observed. Hence, anti-TNFR1 dAbs can be classified based on their ability to inhibit TNFα binding to its receptor 1 in a standard RBA.

One immunoglobulin single variable domain, identified in WO2011051217 as DOM1h-574-208 (SEQ ID NO:2), has been identified by the Applicant as an example of a non-competitive TNFR1-specific binding protein. An example of a competitive TNFR1 binding protein is the heavy chain (Vh) dAb DOM1h-131-206 (SEQ ID NO:3), identified in WO2008149148.

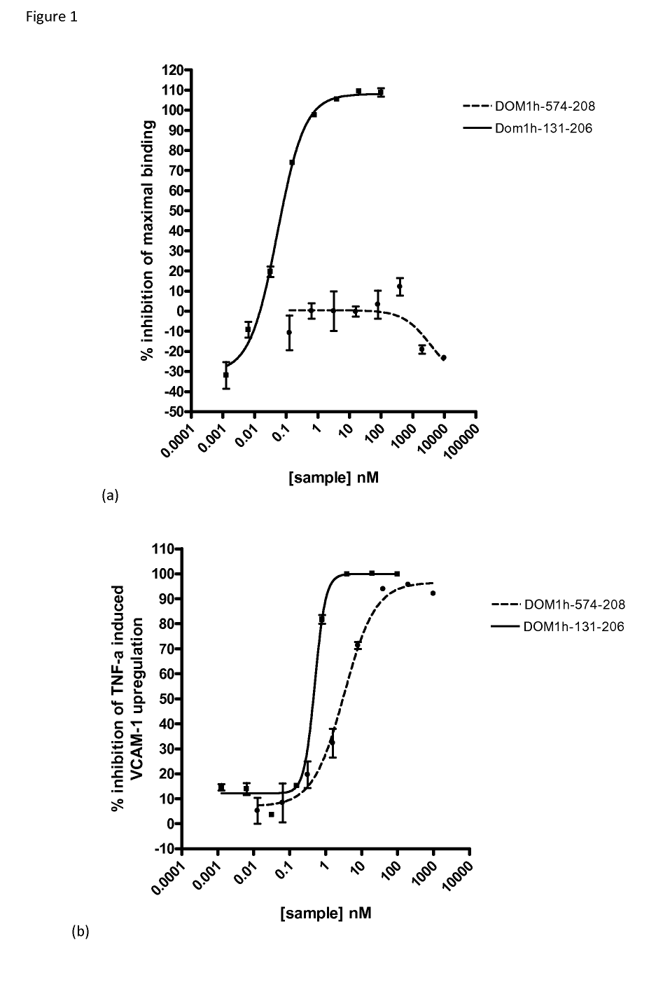

Both dAbs were expressed in E. coli using autoinduction media (OnEx, Novagen) and recombinant protein redirected to the culture media. Both dAbs were purified in a single step using Protein-A streamline (GE Healthcare) and buffer exchanged to PBS for cell assay experiments. As can be seen from FIG. 1(a), the competitive dAb DOM1h-131-206 inhibited TNFα binding to TNFR1 in the RBA while DOM1h-574-208 had no effect on TNFα binding to TNFR1.

However, a dAb which lacks the ability to inhibit the binding of TNFα to its receptor might also lack functional activity in inhibiting TNFα-mediated signalling through TNFR1. Therefore, the RBA should be interpreted together with a cell assay in which dAb-mediated inhibition of a functional response can be investigated. The specific cell assay that was used is a human umbilical vein endothelial cell (HUVEC) where TNFα-induced upregulation of an adhesion marker, vascular adhesion marker-1 (VCAM-1) is used as a marker of TNF-α induced cell activation.

Briefly, in this assay human umbilical vein endothelial cells were plated and pre-incubated with a dose range of anti-TNFR1 dAbs followed by addition TNFα (1 ng/ml). After an 18 h incubation at 37° C. with TNFα, the culture supernatant was aspirated and the cells were lysed. VCAM-1 levels were determined by adding the cell lysates to a VCAM-1 sandwich ELISA. dAbs which are functionally active in the assay will inhibit TNFα-mediated signalling and consequently reduce the level VCAM-1 upregulation by the HUVEC in response to the TNFα stimulation. As can be seen in FIG. 1(b), both competitive dAb (DOM1h-131-206) and the non-competitive anti-TNFR1 dAb (DOM1h-574-208) are able to inhibit TNFα-mediated signalling and are therefore functionally active as TNFα inhibitors.

### Example 2

### Summary of Epitope Mapping with TNFR1 Binding Protein DMS5541

DMS5541 comprises, as a TNFR1 binding protein, the TNFR1 dAb DOM1h-574-208 (SEQ ID NO:2), coupled to a human serum albumin (HSA) binding dAb by a short linker (Ala-Ser-Thr). It is described further in WO2011051217. The epitope of this molecule (referred to as DMS5541) on TNFR1 was determined using hydrogen deuterium exchange mass spectrometry.

Methods and principles on using H/D exchange perturbation for epitope mapping are discussed in a review by Hamuro et al J. Biomol. Tech. (2003) 14:171-182; and Coales et al, Rapid Comm. In Mass Spec. (2009) 23(5):639-647.

For the epitope mapping of TNFR1, H/D exchange analysis of the antigen in the presence and absence of DMS5541 was carried out. The regions of TNFR1 which exchange slower in the presence of DMS5541 compared to speed of exchange when the binding protein is absent is considered to define the epitope on TNFR1. To identify the epitope one requires firstly the identification of proteolytic fragments of the antigen and secondly the determination of the perturbation of the H/D exchange reaction. Suitable methods are described, for example, in U.S. Pat. No. 6,291,189, U.S. Pat. No. 6,331,400 and U.S. Pat. No. 7,280,923.

Data from H/D exchange indicated that the TNFR1-binding domain of DMS5541 bound to a peptide comprising amino acid residues 165 to 172 of TNFR1 (SEQ ID NO:1). Sequence coverage was incomplete making further characterisation of the DMS5541 epitope difficult.

### Example 3

### Elucidation of the TNFR1/TNFα/DOM1h-574-208 Complex

Co-crystallography of DOM1h-574-208 (SEQ ID NO:2) with TNFR1 and TNFα, refined at a resolution of 2.9 Å, now confirms that DOM1h-574-208 binds predominantly to residues within Domain 4 of TNFR1 (crystallographic data collection and model refinement statistics are given below).

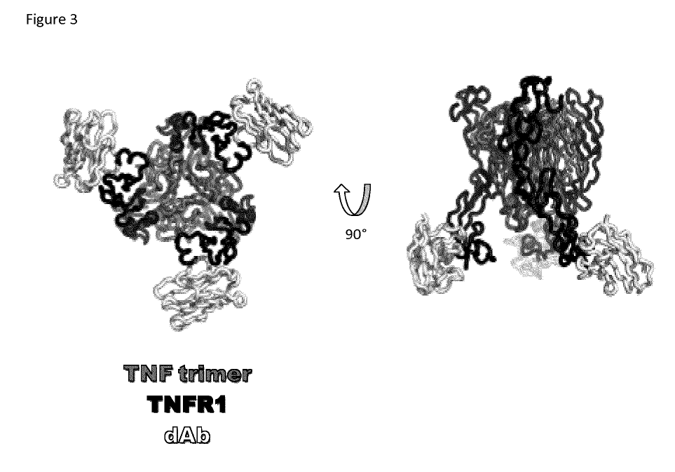

Moreover, the crystallography also reveals that the TNFα ligand is indeed trimeric, and that the TNFα-TNFR1-DOM1h-574-208 complex is also trimeric, forming around, and driven by, the trimeric ligand molecule. The structure is shown graphically in FIG. 3. This is the believed to be the first time the TNFα-TNFR1 structure has been fully described and experimentally isolated.

The structure reveals DOM1h-574-208 as binding to an epitope on the opposite side of the TNFαbinding site on TNFR1. Accordingly, DOM1h-574-208, and other TNFR1 binding molecules which bind in the same area as DOM1h-574-208, cannot disrupt the formation of the TNFα-TNFR1 trimeric complex. Thus, such molecules are non-competitive with TNFα. The complex illustrated in FIG. 3 could form on the cell surface.

Data collection and processing statistics

| X-ray source | PXI/X06SA | (SLS ¹) |
|---|---|---|
| Wavelength [Å] | 1.0000 | |
| Detector | PILATUS 6M | |
| Temperature [K] | 100 | |
| Space group | H 3 2 | |
| Unit cell [Å] | a = 141.38 b = 141.38 c = 88.70 | |
| | α = β = 90.0° γ = 120.0° | |
| Resolution [Å] | 2.89 | (3.12-2.89) |
| Unique reflections | 24583 | (5057) |
| Multiplicity | 3.9 | (3.9) |
| Completeness [%] | 98.2 | (98.9) |
| R_{sym} [%] ³ | 3.8 | (39.8) |
| Rₘₑₐₛ [%] ⁴ | 5.0 | (52.7) |
| I/σ(I) | 9.2 | ((1.9) |
| Mean(I)/sd ⁵ | 16.8 | (2.7) |

¹ SWISS LIGHT SOURCE (SLS, Villigen, Switzerland)
² Values in parenthesis refer to the highest resolution bin
${ }^{3} Rsym = \frac{{\sum }_{h}^{ } {\sum }_{i}^{{n}_{k}} \vert {\hat{I}}_{k} - {I}_{h , i} \vert}{{\sum }_{h}^{ } {\sum }_{i}^{{n}_{h}} {I}_{h , i}} with {\hat{I}}_{k} = \frac{1}{{n}_{h}} {\sum }_{i}^{{n}_{h}} {I}_{h , i}$ where I_{h,i} is the intensity value of the ith measurement of h
${ }^{4} Rmeas = \frac{{\sum }_{h} \sqrt{\frac{{n}_{h}}{{n}_{h} - 1}} {\sum }_{i}^{{n}_{h}} \vert {\hat{I}}_{h} - {I}_{h , i} \vert}{{\sum }_{h}^{ } {\sum }_{i}^{{n}_{k}} {I}_{h , i}} with {\hat{I}}_{k} = \frac{1}{{n}_{h}} {\sum }_{i}^{{n}_{k}} {I}_{h , i}$ where I_{h,i} is the intensity value of the ith measurement of h
⁵ Calculated from independent reflections

Refinement statistics ¹

| | |
|---|---|
| Resolution [Å] | 112.72-2.89 |
| Number of reflections (working/test) | 23892/690 |
| R_{cryst} [%] | 23.4 |
| R_{free}[%] ² | 28.0 |
| Total number of atoms: | |
| Protein | 6385 |
| Water | — |
| 1,2-Ethanediol | 4 |
| Deviation from ideal geometry ³ | |
| Bond lengths [Å] | 0.008 |
| Bond angles [°] | 1.17 |
| Bonded B's [Å²] ⁴ | 1.7 |
| Ramachandran plot ⁵ | |
| Most favoured regions [%] | 83.2 |
| Additional allowed regions [%] | 15.4 |
| Generously allowed regions [%] | 1.3 |
| Disallowed regions [%] | 0.1 |

¹ Values as defined in REFMAC5, without sigma cut-off
² Test-set contains 2.8% of measured reflections
³ Root mean square deviations from geometric target values
⁴ Calculated with MOLEMAN
⁵ Calculated with PROCHECK

### Example 4

### Identification of the DOM1h-574-208 Epitope and Dimerisation Interface

The elucidation of the crystal structure has also enabled the Applicant to further characterise—and add therapeutic relevance to—the specific residues responsible for TNFR1 self-association, and to characterise the specific residues which (a) are involved in TNFα-TNFR1 interaction and (b) form the epitope of DOM1h-574-208.

Residue contacts between various chains in the asymmetric unit (ASU) were calculated by searching for residues within 4.0 Å distance cut-off. Electron density maps and the resulting structural model allow determination of ligand-receptor binding sites and DOM1h-574-208 epitope/paratope. Due to variations in electron density coverage and thus side-chain conformations between the two trimeric complexes which exist in the ASU, there are slight variations in residue contact calculations.

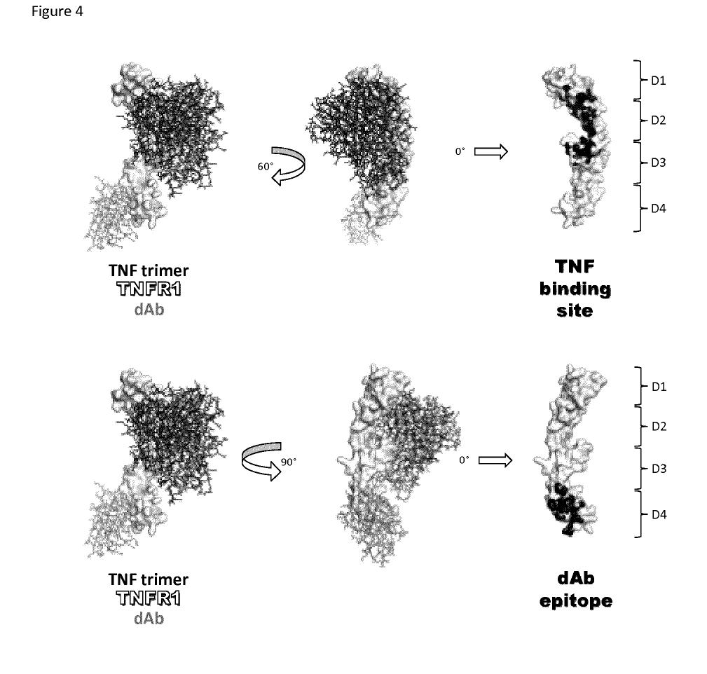

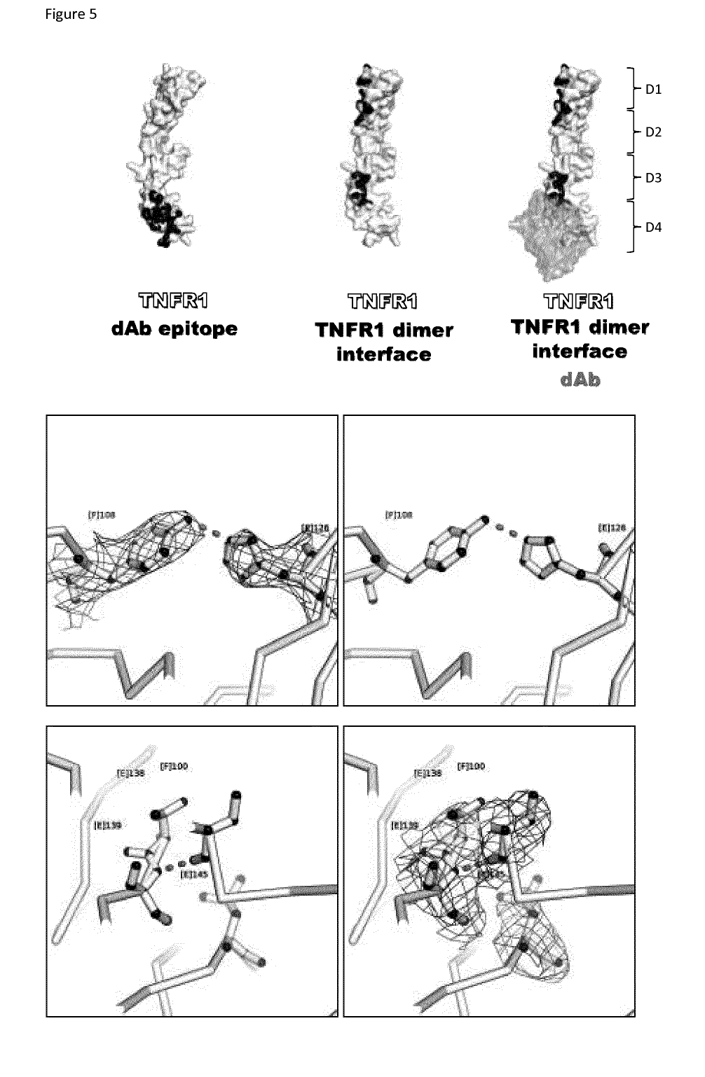

The deduced structure clearly shows non-overlapping binding sites on TNFR1 for TNFα and DOM1h-574-208, supporting the conclusion that it is non-competitive with TNFα. TNFα binds predominantly to domain 2 and DOM1h-574-208 to domain 4 (FIG. 4). The residues involved in the formation of the parallel TNFR1 dimer are shown graphically in FIG. 5, which also shows the overlap of the DOM1h-574-208 epitope and dimerisation interface. The specific residues involved in these interactions are shown in Table 1 below.

TABLE 1

| | Residue within TNFR1 | | |
|---|---|---|---|
| Residue within TNFR1 | (SEQ ID NO: 1) | | |
| (SEQ ID NO: 1 | comprising the epitope | Residue within TNFR1 | |
| comprising the | of DOM1h-574-208 | (SEQ ID NO: 1) involved | Domain with TNFR1 |
| dimerisation interface | (SEQ ID NO: 2) | in TNFα binding | (SEQ ID NO: 1) |
| Q17 | | | Domain 1 |
| G18 | | | |
| K19 | | | |
| T31 | | | |
| K32 | | | |
| C33 | | | |
| H34 | | | |
| K35 | | | |
| G36 | | | |
| T37 | | | |
| | | Y38 | |
| G47 | | | |
| Q48 | | | |
| D49 | | | |
| E54 | | | Domain 2 |
| | | E56 | |
| | | S57 | |
| | | G58 | |
| | | S59 | |
| | | A62 | |
| | | S63 | |
| E64 | | | |
| | | N65 | |
| | | H66 | |
| | | L67 | |
| | | (R68) | |
| | | H69 | |
| | | C70 | |
| | | L71 | |
| | | S72 | |
| | | C73 | |
| | | S74 | |
| | | (K75) | |
| | | R77 | |
| | | (K78) | |
| | | E79 | |
| | | M80 | |
| V90 | | | |
| V91 | | | |
| | | W107 | Domain 3 |
| | | S108 | |
| | | L111 | |
| | | Q113 | |
| | T124 | | |
| H126 | H126 | | |
| L127 | | | |
| Q130 | | | |
| Q133 | | | |
| V136 | | | |
| T138 | T138 | | |
| | C139 | | Domain 4 |
| | H140 | | |
| | A141 | | |
| | G142 | | |
| | F143 | | |
| | F144 | | |
| L145 | L145 | | |
| | E161 | | |
| | L165 | | |
| | L167 | | |
| | P168 | | |
| | Q169 | | |

[symbol]

As mentioned above, the asymmetric unit (ASU) contains two copies of the trimeric complex of TNFα:TNFR1:dAb. Residues elucidated to be on the contact surfaces (within 4.0 Å) of either chain are shown in Table 2; The level of electron density defining side-chains differs between non-crystallographic symmetry related protein chains; those in bold font are elucidated as contact surface residues on the basis of electron density data from both chains in the ASU. [symbol] [symbol]
Residues shown in parenthesis are Arginine or Lysine residues which are not defined by electron density but fall within the 4.0 Å contact region in the final refined model. These residues are included in Table 2 as they are believed to be within 4.0 Å on the basis of the elucidated model.

### Example 5

### Prevention of TNFα-TNFR1 Multimerisation

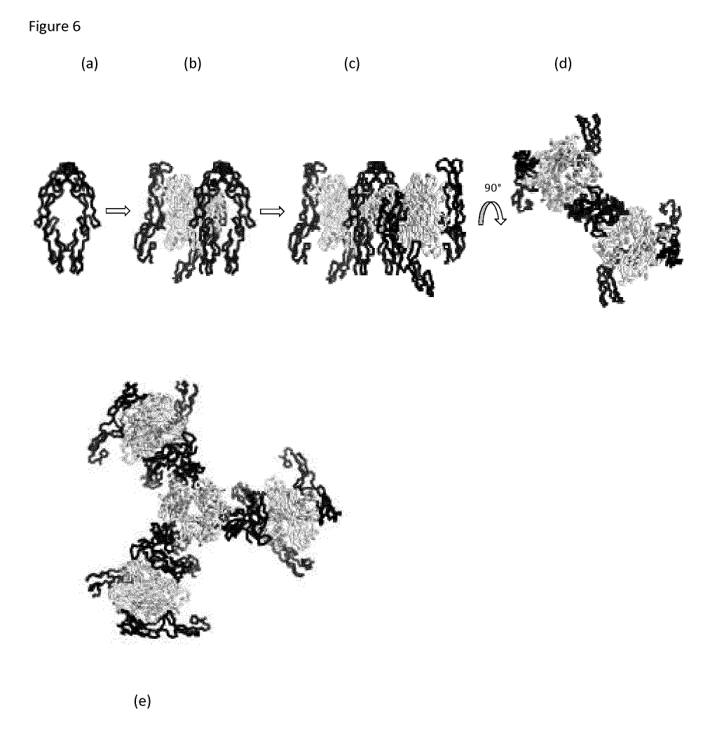

TNFα activates signaling by trimerisation of TNFR1 and signal amplification is thought to occur by multimerisation of trimeric ligand-receptor complexes on the cell surface. This multimerisation event can be modeled based on the elucidated structure of the TNFR1/TNFα/DOM1h-574-208 complex (FIG. 6). The dimerisation interface is distinct from the TNFα binding region and thus trimeric ligand receptor complexes could multimerise by TNFR1 dimerisation as illustrated in FIG. 6 below.

FIG. 6 shows a step-wise formation of the multimerised TNFα-TNFR1 complex. FIG. 6(a) is an image of TNFR1 showing how the receptor can exist as a parallel dimer (Naismith et al, ibid.). FIG. 6(b) and (c) show the coming together of two TNFα-TNFR1 trimers associated by the dimerised TNFR1 chains, seen rotated 90 degrees clockwise in (d). (e) illustrates how TNFα-TNFR1 trimers can further multimerise (viewed down the 3-fold symmetry axis of the central TNFα trimer), with the potential to further amplify downstream signalling.

### Example 6

### Prevention of the Formation of Multimerised TNFα-TNFR1 Trimeric Complexes

As shown in Examples 1 and 2, binding of DOM1h-574-208 is non-competitive with TNFα, and binding is to an epitope on the opposite side of the receptor chain to the TNFα binding site. Therefore, binding of the DOM1h-574-208 dAb to TNFR1 cannot disrupt the formation of the TNFα-TNFR1 trimer.

Binding of the DOM1h-574-208 dAb would prevent multimerisation of the TNFα-TNFR1 complexes as the dAb binds to a region predominantly in domain 4 which forms part of the TNFR1 dimerisation interface (FIG. 5). As the basis for the formation of stable trimeric ligand-receptor multimers is dimer formation by TNFR1, the binding of an TNFR1 binding protein to the dimerisation interface would block receptor signal amplification by preventing the formation of such multimers. The binding to this interface will not affect formation of the ligand-receptor trimer (i.e. non-multimerised conformation) and this would continue to signal weakly. The model is depicted graphically in FIG. 7. The arrow identifies the region of steric hindrance in (a).

FIG. 7(b) schematically represents the interaction of TNFR1 with TNFR1 binding proteins, in the presence and absence of its natural ligand.

Panel A: TNFR1 exists on cell surface mainly as a dimer which can dissociate and form complexes with domain antibody or bivalent domain antibody-Fc via an epitope located in the dimerisation interface region. Neither of these interactions activates the receptor. TNFR1 dimer cross-linking through a TNF-α binding site though will trigger signalling of the receptor.

Panel B: The various complexes of TNFR1 can also interact with TNF-α. Although only two of the three binding sites are shown as occupied, the third one too is envisaged to be available to a similar interaction. In the case of domain antibody/TNFR1 complexes their cross-linking by TNF-α is insufficient to trigger signalling. However, TNF-α cross-linking of a complex of domain antibody-Fc with TNFR1 triggers signalling. It is proposed that in a minimal TNFR1 signalling complex it is the interaction between a receptor-bound chain of TNFR1 dimer with a non-receptor bound chain of the TNFR1 that is required for signalling, presumably as a result of favourably oriented intracellular death domains or any associated proteins. This model also supports in vitro cell assay data where weak signaling is observed in the presence of DOM1h-574-208 (Example 1).

TNFR1 binding proteins which bind to the TNR1 dimerisation interface regions, in particular, the TNFR1 interface region in Domains 3 and 4, would therefore be expected to function in the same manner as DOM1h-574-208.

### SEQUENCE INFORMATION

SEQ ID NO: 1 - Polypeptide sequence of human TNFR1 (extracellular region)
LVPHLGDREKRDSVCPQGKYIHPQNNSICCTKCHKGTYLYNDCPGPGQDTDCRECESGSFTASENHLRHCLSCSKC
RKEMGQVEISSCTVDRDTVCGCRKNQYRHYWSENLFQCFNCSLCLNGTVHLSCQEKQNTVCTCHAGFFLRENECV
SCSNCKKSLECTKLCLPQIENVKGTEDSGTT
SEQ ID NO: 2 - Polypeptide sequence of DOM1h-576-208
EVQLLESGGGLVQPGGSLRLSCAASGFTFDKYSMGWVRQAPGKGLEWVSQISDTADRTYYAHAVKGRFTISRDNS
KNTLYLQMNSLRAEDTAVYYCAIYTGRWVPFEYWGQGTLVTVSS
SEQ ID NO: 3 - Polypeptide sequence of DOM1h-131-206
EVQLLESGGGLVQPGGSLRLSCAASGFTFAHETMVWVRQAPGKGLEWVSHIPPDGQDPF
YADSVKGRFTISRDNSKNTLYLQMNSLRAEDTAVYHCALLPKRGPWFDYWGQGTLVTVSS
SEQ ID NO: 4 - Polypeptide sequence of TNFα
VRSSSRTPSDKPVAHVVANPQAEGQLQWLNRRANALLANGVELRDNQLVVPSEGLYLIYSQVLFKGQGCPSTHVL
LTHTISRIAVSYQTKVNLLSAIKSPCQRETPEGAEAKPWYEPIYLGGVFQLEKGDRLSAEINRPDYLDFAESGQVYFGII
AL

1-18. (canceled)

19. A TNFR1 binding protein which binds to an epitope within Domains 3 and/or 4 of TNFR1 and prevents dimerisation of TNFR1, wherein the epitope does not comprise or require residues H126, T138 or L145 of TNFR1 (SEQ ID NO:1).

20. A TNFR1 binding protein, wherein the TNFR1 binding protein binds to an epitope on TNFR1 (SEQ ID NO:1), wherein the epitope comprises or consists of one or more residues selected from: E54, E64, V90, V91, H126, L127, Q130, Q133, V136, T138 and L145 of SEQ ID NO:1, and wherein, if the TNFR1 binding protein binds to an epitope that comprises or consists of one or more of residues H126, T138 and L145, the TNFR1 binding protein is not an immunoglobulin single variable domain.

21. A TNFR1 binding protein according to claim 19, which inhibits TNFR1 dimerisation.

22. A TNFR1 binding protein according to claim 19, wherein the TNFR1 binding protein binds to an epitope on TNFR1 (SEQ ID NO:1) comprising one or more residues selected from: E54, E64, V90, V91, L127, Q130, Q133 and V136 of SEQ ID NO:1.

23. A TNFR1 binding protein according to claim 19, wherein the epitope comprises one or more residues selected from: E54, E64, V90 and V91 of SEQ ID NO:1.

24. A TNFR1 binding protein according to claim 19, wherein the epitope comprises one or more residues selected from: L127, Q130, Q133 and V136 of SEQ ID NO:1.

25. A TNFR1 binding protein according to claim 20, which is an antagonist of TNFR1.

26. A TNFR1 binding protein according to claim 25, which is a non-competitive antagonist of TNFR1.

27. A TNFR1 binding protein according to claim 19, which monovalently binds to TNFR1.

28. A TNFR1 binding protein according to claim 19, which is an immunoglobulin single variable domain.

29. A TNFR1 binding protein according to claim 19, wherein said binding protein has a second binding specificity for an antigen other than TNFR1.

30. A TNFR1 binding protein according to claim 29, wherein said antigen is serum albumin.

31. A method of screening for non-competitive antagonists of TNFR1, comprising the steps of providing a plurality of TNFR1 binding proteins, determining the ability of said TNFR1 binding proteins to antagonise TNFR1 signalling; determining the ability of said TNFR1 binding proteins to disrupt the binding of TNFR1 to TNFα; and selecting those TNFR1 binding proteins which antagonise TNFR1 but which do not disrupt the binding of TNFR1 to TNFα.

32. A method of screening for non-competitive antagonists of TNFR1, comprising the steps of determining the epitope of a TNFR1 antagonist, and selecting antagonists which have an epitope comprising one or more amino acid residues of TNFR1 (SEQ ID NO:1) selected from: E54, E64, V90, V91, H126, L127, Q130, Q133, V136, T138 and L145.

33. A method of preventing dimerisation of TNFR1, comprising the steps of providing a TNFR1 binding protein according to claim 19 under conditions suitable to allow it to bind to TNFR1, thereby preventing the TNFR1 chain from dimerisation.

34. A composition comprising a TNFR1 binding protein according to claim 19 in a physiologically acceptable carrier.

35. A method for the treatment or prophylaxis of an inflammatory condition in a patient, the method comprising administering the composition of claim 34, to the patient.


SEQUENCE LISTING (4 sequences)

SEQ ID NO: 1 - protein, 183 aa, Homo Sapiens
PLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTT

SEQ ID NO: 2 - protein, 120 aa, Artificial Sequence
  Single Variable Domain
PEVQLLESGGGLVQPGGSLRLSCAASGFTFDKYSMGWVRQAPGKGLEWVSQISDTADRTYYAHAVKGRFTISRDNSKNTLYLQMNSLRAEDTAVYYCAIYTGRWVPFEYWGQGTLVTVSS

SEQ ID NO: 3 - protein, 120 aa, Artificial Sequence
  Single Variable Domain
PEVQLLESGGGLVQPGGSLRLSCAASGFTFAHETMVWVRQAPGKGLEWVSHIPPDGQDPFYADSVKGRFTISRDNSKNTLYLQMNSLRAEDTAVYHCALLPKRGPWFDYWGQGTLVTVSS

SEQ ID NO: 4 - protein, 158 aa, Homo Sapiens
PVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL